(19) United States
(12) Patent Application Publication
(10) Pub. No.: US 2024/0418705 A1
(43) Pub. Date: Dec. 19, 2024

(54) CELLULAR POPULATIONS AND USES THEREOF

(71) Applicant: The Council of the Queensland Institute of Medical Research, Herston (AU)

(72) Inventors: Geoffrey R. Hill, Herston (AU); Ping Zhang, Herston (AU)

(21) Appl. No.: 18/746,738

(22) Filed: Jun. 18, 2024

Related U.S. Application Data
(63) Continuation of application No. 18/298,129, filed on Apr. 10, 2023.
Application No. 18/298,129 is a continuation of application No. 16/603,454, filed on Oct. 7, 2019, now Pat. No. 11,662,343.

(30) Foreign Application Priority Data
Apr. 7, 2017 (AU) 2017901292

(51) Int. Cl. G01N 33/50; A61K 39/00; C07K 14/535; C07K 14/54; C07K 14/73; C12N 5/0783

(52) U.S. Cl. CPC G01N 33/505; A61K 39/461; A61K 39/4611; A61K 39/4621; A61K 39/46434; A61K 2239/26; A61K 2239/38; C07K 14/535; C07K 14/5412; C07K 14/5428; C07K 14/5446; C07K 14/70514; C12N 5/0637

(57) ABSTRACT

Disclosed are methods of identifying immunosuppressive T_{R}1 regulatory T cells, including methods of diagnosing the presence of immune tolerance, methods of producing immunosuppressive regulatory T cells, and methods of eliciting immune tolerance in a subject. These methods include screeing T cells to detect Eomes ⁺IL-10⁺ T cells or expressing recombinant Eomes in T cell populations to generate immunosuppressive regulatory T cells.

### FIELD OF THE INVENTION

This application is a continuation of U.S. patent application Ser. No. 18/298,129, filed Apr. 10, 2023, which is a continuation of U.S. patent application Ser. No. 16/603,454, filed Oct. 7, 2019, now U.S. Pat. No. 11,662,343, which is a § 371 National Entry Application of PCT/AU2018/050322, filed Apr. 9, 2018, each of which are incorporated herein by reference in their entirety. The application claims priority to Australian Provisional Application No. 2017901292 entitled “Cellular populations and uses therefor” filed Apr. 7, 2017.

This invention relates generally to methods of identifying immunosuppressive T_{R}1 regulatory T cells, including in methods of diagnosing the presence of immune tolerance, methods of producing immunosuppressive regulatory T cells, and methods of eliciting immune tolerance in a subject.

Bibliographic details of certain publications numerically referred to in this specification are collected at the end of the description.

### BACKGROUND OF THE INVENTION

Type-1 regulatory T (T_{R}1) cells are a FoxP3 negative, IL-10 producing T cell population, which have potent immune suppressive functions and bear alloantigen specificity (1, 2). IL-10 is the major mediator by which T_{R}1 cells assert their immunomodulatory role. Direct and bystander-mediated T cell suppression by TGFβ and granzyme B-dependent killing of antigen presenting cells (APC) have also been described (reviewed in (3)). In addition to IL-10, T_{R}1 cells show high expression of TGF-β, secrete intermdiate amounts of IFNγ but no IL-2 or IL-4 (3-5). Extensive studies have demonstrated the importance of T_{R}1 cells in maintaining immune tolerance or limiting overt inflammation after transplantation, during autoimmune disease or after infections (6-9). IL-27 has been identified as a main driver of T_{R}1 cell differentiation via the activation of transcription factors that include B-lymphocyte-induced maturation protein-1 (Blimp-1), the aryl hydrocarbon receptor (AhR) and c-Maf (5-8, 10-12). However, the function, phenotype and lineage development of T_{R}1 cells in disease states remains poorly understood (5, 13).

Graft-versus-host disease (GVHD) is a common complication of allogeneic bone marrow transplantation (BMT), limiting survival and quality of life (14). CD4⁺ FoxP3⁺ regulatory T (T_{reg}) cells are a well defined regulatory population important for the generation of tolerance after BMT (15). Due to impaired homeostasis of T_{reg} cells after allogeneic BMT (16), other suppressive cell populations such as T_{R}1 cells may be imperative for the prevention and treatment of GVHD. Consistent with this idea, IL-10 deficiency in donor T cells results in more severe GVHD (17, 18).

### SUMMARY OF THE INVENTION

In work leading up to the present invention, a mouse model was developed using a dual Il10^{GFP}/Foxp3^{RFP} reporter mouse strain (19, 20) to delineate regulatory T cell responses after experimental BMT. Using GVHD as a disease model, it was found that T_{R}1 cells are the most abundant IL-10 producing regulatory T cell population after experimental BMT. Further analyses demonstrated unexpectedly that T_{R}1 cells that develop during GVHD express high amounts of Eomes, which is required for their development and that over-expression of Eomes promotes T_{R}1 cell development both in vivo and in vitro. Eomes acts in concert with Blimp-1, a known transcriptional regulator of T_{R}1 cell differentiation (6-8, 21), to induce IL-10 expression. The present inventors also found that Eomes expression and T_{R}1 cell development require T-bet and donor macrophage-derived IL-27, resulting in a T-bet^{lo}Eomes^{hi} phenotype. Additionally, it was found that Eomes⁺ T_{R}1 cells are abundant after clinical BMT. These findings permit the development of new therapeutic strategies in detecting immunosuppressive T_{R}1 cells, including the presence of immune response that is anergic or tolergenic, and in eliciting immune tolerance, as described hereafter.

Accordingly, in one aspect, the present invention provides methods of identifying immunosuppressive T_{R}1 regulatory T cells in a sample. These methods generally comprise: screening T cells in the sample to detect Eomes⁺IL-10⁺ T cells; and identifying the detected T-cells as immunosuppressive T_{R}1 regulatory T cells. Suitably the methods further comprise isolating the identified immunosuppressive regulatory T-cells. In some embodiments, the screening step is further characterized by detection of Eomes^{hi} T cells, IL-10^{hi} T cells or Eomes^{hi}IL-10^{hi} T cells. In some of the same and other embodiments, the screening step is further characterized by detection of T-bet^{lo}Eomes⁺IL-10⁺ T cells. In some of the same and other embodiments, the screening step is further characterized by detection of Eomes⁺IL-10⁺ T cells that are positive or high for IFNγ. In some of the same and other embodiments, the screening step is further characterized by detection of Eomes⁺IL-10⁺ T cells that are positive for at least one (e.g., 1, 2, 3, 4, 5, 6) of CD4, CD122, α4β7, LAG-3, Ly6C and TIGIT, and/or negative or low for one or more (e.g., 1, 2, 3) of CD25, CD69 and FoxP3. In some of the same and other embodiments, the screening step is further characterized by detection of Eomes⁺IL-10⁺ T cells that are negative or low for T_{H}2 cytokines such as IL-4, IL-13 and IL-5, and/or for T_{H}17 cytokines such as IL-17, IL-6 and GM-CSF. Suitably, the screening methods further comprise detecting suppression by the Eomes⁺IL-10⁺ T cells of at least one immune function selected from the group consisting of IL-2 production, cell proliferation, cytokine production, cell migration, and effector functions, killing, and T-cell proliferation. The sample may be a peripheral blood mononuclear cell (PBMC) sample or a lymphoid tissue sample.

In a related aspect, the present invention provides methods of diagnosing the presence of immune tolerance in a subject. These methods generally comprise detecting the presence in the subject of Eomes⁺IL-10⁺ T cells as broadly described above and elsewhere herein to thereby diagnose the presence of immune tolerance in the subject. Suitably, the methods comprise detecting the presence of Eomes⁺IL-10⁺ T cells in a sample obtained from the subject. In specific embodiments, the immune tolerance is an antigen-specific immune tolerance. In illustrative examples of this type, the antigen is associated with a disease or disorder such as but not limited to an inflammatory disorder, a cancer, an autoimmune disorder (e.g., type 1 diabetes, rheumatoid arthritis, Systemic Lupus Erythematosus (SLE), multiple sclerosis, or myasthenia gravis), a graft vs. host disease, an organ transplantation rejection, an allergy, allergic rhinitis, a food allergy or asthma.

Another aspect of the present invention provides methods of producing an isolated population of immunosuppressive regulatory T cells. These methods generally comprise: isolating a heterogenous population of cells (e.g., PBMC) comprising regulatory T cells; optionally enriching for T cells that are positive or high for at least one (e.g., 1, 2, 3, 4, 5, 6) of CD4, CD122, α4β7, LAG-3, Ly6C and TIGIT, and/or negative or low for one or more (e.g., 1, 2, 3) of CD25,-2-CD69 and FoxP3; and expressing Eomes in the T cells to thereby make an isolated population of immunosuppressive regulatory T cells. Suitably, these methods further comprise isolating Eomes⁺IL-10⁺ T cells from the heterogenous population or enriched T cell population. In specific embodiments, the production methods comprise introducing into the T cells a construct that comprises an Eomes coding sequence in operable connection with a regulatory sequence that is operable in the T cells. In some of the same and other embodiments, the production methods further comprise expanding the population, for example, by contacting the isolated T cells of the population with antigen, alloantigen, and/or anti-CD3/anti-CD28 antibodies plus IL-2 in the presence of TGFβ and/or IL-27. In representative examples of this type, the Eomes⁺IL-10⁺ T cells are antigen-specific immunoregulatory T cells.

In a related aspect, the present invention provides methods of producing an immunosuppressive regulatory T cell. These methods generally comprise, consist or consist essentially of: expressing in a T cell that is positive or high for at least one (e.g., 1, 2, 3, 4, 5, 6) of CD4, CD122, α4β7, LAG-3, Ly6C and TIGIT, and/or negative or low for one or more (e.g., 1, 2, 3) of CD25, CD69 and FoxP3, a recombinant Eomes coding sequence to thereby produce an immunosuppressive regulatory T cell. Suitably, the T cell is positive or high for CD4. Suitably, the immunosuppressive regulatory T cell is an IL-10⁺CD4⁺ T cell. In some embodiments, the immunosuppressive regulatory T cell is an Eomes^{hi} T cell. In some of the same and other embodiments, the immunosuppressive regulatory T cell is a T-bet^{lo} T cell. In some of the same and other embodiments, the immunosuppressive regulatory T cell is an IFNγ+ T cell. In representative examples, the immunosuppressive regulatory T cell is high for one or both of IL-10 and IFNγ. In some of the same and other embodiments, the immunosuppressive regulatory T cell is negative or low for any one or more T_{H}2 cytokines such as IL-4, IL-13 and IL-5, and/or any one or more T_{H}17 cytokines such as IL-17, IL-6 and GM-CSF. Suitably, the immunosuppressive regulatory T cell is capable of suppressing at least one immune function selected from the group consisting of IL-2 production, cell proliferation, cytokine production, cell migration, and effector functions, killing, and T-cell proliferation. In specific embodiments, the production methods further comprise introducing into the T cell a construct that comprises an Eomes coding sequence in operable connection with a regulatory sequence that is operable in the T cell. In some of the same and other embodiments, the production methods comprise expressing the recombinant Eomes coding sequence in the absence of contacting the T cell with IL-27 (e.g., in the absence of culturing the T cell with exogenous IL-27 or with an IL-27-producing cell such as an IL-27-producing antigen-presenting cell (e.g., an IL-27-producing DC)). In specific embodiments, the methods further comprise contacting the T cell with an antigen or alloantigen. In some of the same nd other embodiments, the methods further comprise contacting the T cell with one or both of an anti-CD3 antibody and an anti-CD28 antibody.

In another related aspect, the present invention provides an immunosuppressive regulatory T cell comprising, consisting or consisting essentially of a construct that comprises an Eomes coding sequence in operable connection with a regulatory sequence that is operable in the T cell. Suitably the immunosuppressive regulatory T cell is an IL-10⁺CD4⁺ T cell. In some embodiments, the T cell is an Eomes^{hi} T cell. In some of the same and other embodiments, the T cell is a T-bet^{lo} T cell. In some of the same and other embodiments, the T cell is an IFNγ+ T cell. In representative examples, the T cell is high for one or both of IL-10 and IFNγ. In some of the same and other embodiments, the T cell is negative or low for any one or more T_{H}2 cytokines such as IL-4, IL-13 and IL-5, and/or any one or more T_{H}17 cytokines such as IL-17, IL-6 and GM-CSF. Suitably, the immunosuppressive regulatory T cell is capable of suppressing at least one immune function selected from the group consisting of IL-2 production, cell proliferation, cytokine production, cell migration, and effector functions, killing, and T-cell proliferation.

In yet another aspect, methods are provided for eliciting immune tolerance in a subject (e.g., a mammal including a primate such as a human). These methods generally comprise administering to the subject a population of immunoregulatory T cells comprising Eomes⁺IL-10⁺ T cells to thereby elicit immune tolerance in the subject. Suitably the Eomes⁺IL-10⁺ T cells comprise Eomes⁺IL-10⁺CD4⁺ T cells. In some embodiments, the Eomes⁺IL-10⁺ T cells comprise Eomes^{hi} T cells. In some of the same and other embodiments, the Eomes⁺IL-10⁺ T cells comprise T-bet^{lo} T cells. In some of the same and other embodiments, the Eomes⁺IL-10⁺ T cells comprise IFNγ+ T cells. In representative examples, the Eomes⁺IL-10⁺ T cells are high for one or both of IL-10 and IFNγ. In some of the same and other embodiments, the Eomes⁺IL-10⁺ T cells are negative or low for T_{H}2 cytokines such as IL-4, IL-13 and IL-5, and/or T_{H}17 cytokines such as IL-17, IL-6 and GM-CSF. Suitably, the immunosuppressive regulatory T cells are capable of suppressing at least one immune function selected from the group consisting of IL-2 production, cell proliferation, cytokine production, cell migration, and effector functions, killing, and T-cell proliferation.

In specific embodiments, the Eomes⁺IL-10⁺ T cells contain a construct comprising an Eomes coding sequence that is operably connected to a regulatory sequence that is operable in the Eomes⁺IL-10⁺ T cells. In related embodiments, the immune tolerance elicitation methods further comprise introducing the construct into the Eomes⁺IL-10⁺ T cells.

Suitably, the population of immunoregulatory T cells is enriched for Eomes⁺IL-10⁺ T cells. In related embodiments, the immune tolerance elicitation methods further comprise isolating a heterogenous population of cells (e.g., PBMC) and enriching for T cells that are positive or high for at least one (e.g., 1, 2, 3, 4, 5, 6) of CD4, CD122, α4β7, LAG-3, Ly6C and TIGIT, and/or negative or low for one or more (e.g., 1, 2, 3) of CD25, CD69 and FoxP3.

The population of immunoregulatory T cells (e.g., an isolated heterogeneous population or enriched population as described for example above) may be expanded to provide an expanded population of immunoregulatory T cells. Thus, in related embodiments, the immune tolerance elicitation methods further comprise expanding the population, for example, by contacting the isolated T cells of the population with antigen, alloantigen, or anti-CD3/anti-CD28 antibodies plus IL-2 in the presence of TGFβ and/or IL-27. In representative examples of this type, the Eomes⁺IL-10⁺ T cells are antigen-specific immunoregulatory T cells.

The Eomes⁺IL-10⁺ T cells may be autologous or allogeneic to the subject.

Suitably, the subject has an immune or autoimmune disorder (e.g., type 1 diabetes, rheumatoid arthritis, Systemic Lupus Erythematosus (SLE), multiple sclerosis, or myasthenia gravis) and is in need of immunosuppression. In some embodiments, the immune disorder is selected from the group consisting of graft vs. host disease, an organ transplantation rejection, and allergy. In other embodiments, the immune disorder is allergic rhinitis, a food allergy, or asthma.

In specific embodiments, the subject is a recipient of a transplant. The transplant may be recipient (i.e., autologous) or donor-derived (allogeneic or xenogeneic). In specific embodiments, the transplant is a bone marrow transplant. Suitably, the immune system of the subject is not systemically suppressed. In representative examples of ths type, the immunoregulatory T cells are administered concurrently with exposure of the subject to the transplant. In some embodiments, the immunoregulatory T cells are administered simultaneously with exposure of the subject to the transplant. In other embodiments, the immunoregulatory T cells are administered before transplant exposure (e.g., within 1, 2, 3, 4, 5, 6, 7 days) or after transplant exposure (e.g., after 1, 2, 3, 4, 5, 6, 7 days).

In a related aspect, the present invention provides methods for attenuating or inhibiting the development of graft-versus-host disease (GVHD) in a subject receiving a graft. These methods generally comprise administering to the subject a population of immunoregulatory T cells comprising Eomes⁺IL-10⁺ T cells to thereby attenuate or inhibit the development of GVHD in the subject.

### BRIEF DESCRIPTION OF THE DRAWINGS

FIGS. 1A-I are graphical representations showing that T_{R}1 cells constitute the major regulatory T cell after allogeneic BMT. (A-E) B6 (Syn) and B6D2F1 (Allo) mice were transplanted with B6 CD3⁺ T (Il10^{GFP}/Foxp3^{RFP}). (A) Gating strategy after BMT for analysis and FACS sorting of T_{R}1 (red), T_{reg} (blue) and T_{con} (green) cells. (B) Schema of BMT. (C) Expression of IL-10 and FoxP3 in the spleen at d14 (representative of >3 experiments). (D) Frequencies of T_{R}1 and T_{reg} cells at d 14 (Il10^{GFP+}: solid bar; Il10^{GFPneg}: open bar). (E) CD4⁺ T cell subsets in spleen after BMT (n=8−9 per group each time point). (F) B6D2F1 mice were transplanted with B6 CD4⁺ T (Il10^{GFP} and Foxp3^{RFP}) and frequencies of T_{R}1 and T_{reg} cells in the spleen at d14 (n=14). (G) Suppression of proliferation of CFSE labelled B6 CD4⁺ and CD8⁺ responder T cells in vitro by naïve T_{reg} cells versus T_{R}1, T_{reg} and T_{con} “suppressors” sorted from 10 transplant recipients at d14 (data combined from 2 experiments). (H) Experimental BMT schema showing adoptive transfer of sorted T_{R}1 cells to treat established acute GVHD and (I) survival of recipients are shown (n=8 in TCD group, others n=11-12). Data represents mean±SEM.

FIGS. 2A-C are graphical representations showing that T_{R}1 cells express Eomes and display a distinct phenotypic profile. (A-C) B6D2F1 mice were transplanted with Il10^{GFP}Foxp3^{RFP} B6 CD3⁺ T cells. CD4⁺ T cells from spleen were FACS sorted into T_{R}1, T_{reg} and T_{con} cells at d14 as in FIG. 1A. (Data from 3 experiments, ND=not detectable) (A) Cytokine production in culture supernatant of T cell subsets. (B) Expression of transcription factors in T cell subsets (T_{R}1: red, T_{reg}: blue, T_{con}: green, isotype: gray). (C) Expression of cytokines and Eomes in T cell subsets. Data represents mean±SEM.

FIGS. 3A-D are graphical representations showing that Eomes is required for T_{R}1 cell differentiation. (A-D) B6D2F1 mice were transplanted with primary or retrovirally transduced (Mock-GFP or Eomes-GFP) CD4⁺ T cells. (A) Expression of IL-10, IFNγ, FoxP3, Eomes and T-bet (Eomes⁺IL-10⁺: open bar; Eomes^{neg}IL-10⁺: solid bar, n=10 per group) in recipients of WT or Eomes^{-/-} CD4⁺ T cells at d14 (n=10 per group). (B) Expression of IL-10, IFNγ, and Eomes in transduced WT or Eomes^{-/-} CD4⁺ T cells at d7 (n=8 per group) and (C) transcription of Il10 and related genes (data are from 4-5 pooled animals in triplicate reactions, representative of 2 independent experiments). (D) CD4⁺ T cells or Foxp3^{RFPneg}/10^{GFP+}T_{R}1 cells were FACS sorted from spleen and liver at d14 (representative of 3 experiments). A schematic diagram of the mouse IL-10 promoter indicates Eomes binding sites upstream of the TSS with each sequence shown. Recruitment of Eomes to the Il10 promoter and control regions in CD4⁺ T cells from T_{R}1 cells (data are from 30 pooled animals in triplicate reactions) and recruitment of RNA Pol II to the Il10 promoter in WT or Eomes^{-/-} CD4⁺ T cells (data are from 10 pooled animals in triplicate reactions). Data represents mean±SEM.

FIGS. 4A-F are graphical representations showing that Eomes⁺ T_{R}1 cells are dependent on Blimp-1, IL-27 and IL-10. (A-F) B6D2F1 mice were transplanted with primary or retrovirally transduced (Mock-GFP or Eomes-GFP) CD4⁺ T cells and spleen examined after BMT. (A) Expression of Eomes, IL-10 and IFNγ in WT or Blimp-1^{-/-} CD4⁺ T cells at d14 (n=14-15 per group). (B) Expression of Eomes, IL-10 and IFNγ (n=18, 17 for WT; n=13, 14 for Blimp-1^{-/-}) in transduced CD4⁺ T cells at d7-10. (C) Recruitment of Eomes to Il10 promoter in transduced CD4⁺ T cells (WT or Blimp-1^{-/-}) at d10 (data are from 4 animals in duplicate or triplicate reactions). (D) Expression of T-bet, Eomes and IL-10 in WT or Il27r^{-/-} CD4⁺ T cells at d14 (n=10 per group). (E) Expression of Eomes and IFNγ⁺IL-10⁺ T_{R}1 cells in WT or Il10r^{-/-} CD4⁺ T cells at d14 (n=10 per group). (F) Expression of Eomes and IL-10 (Eomes⁺IL-10⁺: open bar; Eomes⁺IL-10^{neg}: solid bar) in CD4⁺ T cells in recipients of WT or Il10^{-/-} CD4⁺CD25^{neg} T cells at d14 (n=10−11 per group). Data represents mean±SEM.

FIGS. 5A-E are graphical representations showing attenuation of GVHD by Eomes⁺ T_{R}1 cells. (A-E) B6D2F1 recipients were transplanted with CD4⁺ T cells and survival or histopathology examined. (A) Survival of recipients of WT or Blimp-1^{-/-} CD4⁺ T cells (2×10⁶ per mouse) (n=11 per T cell group, n=7 in TCD; 2 experiments). (B) Survival of recipients of WT or Il27r^{-/-} CD4⁺CD25^{neg} T cells (2×10⁶ per mouse) (n=12 per T cell group, n=7 in TCD; 2 experiments). (C and D) Histology in recipients of (C) WT versus Il10^{-/-} or (D) WT versus Il10^{fl/fl}×Lck-cre CD4⁺CD25^{neg} T cells (1×10⁶ per mouse) at d28 (n=6 per T cell group, n=3 in TCD group). (E) Survival of recipients of WT or Eomes^{-/-} CD4⁺CD25^{neg} T cells (1×10⁶ per mouse) (n=12 per T cell group, n=7 in TCD; 2 experiments). Histology represents mean±SEM.

FIGS. 6A-G are graphical representations showing that Eomes and T-bet jointly regulate T_{R}1 cell development. (A and B) B6.WT or B6.Ifngr^{-/-} CD3⁺ T cells were transplanted into B6D2F1 mice and splenic CD4⁺ T cells examined at d14. (A) Representative plots show expression of T-bet and Eomes and (B) frequencies of T_{R}1 and T_{reg} cells and expression of IL-10 and Eomes (n=10 per group). (C) B6.Il10^{GFP}Foxp3^{RFP} CD3⁺ T cells were transplanted into B6D2F1 mice receiving αIFNγ or control mAb and splenic CD4⁺ T cells examined at d12 (n=5 per group). Frequencies of T_{R}1 and T_{reg} cells and expression of Eomes and IL-10 are shown. (D) B6D2F1 mice were transplanted with WT or Tbx21^{-/-} CD4⁺ T cells and expression of transcription factors and cytokines in splenic CD4⁺ T cells at d12 shown (n=10 per group). (E) B6D2F1 mice were transplanted with retrovirally (Mock-GFP or Eomes-GFP) transduced WT or Tbx21^{-/-} CD4⁺ T cells and expression of IL-10, IFNγ, IL-4 and GATA-3 in splenic CD4⁺ T cells at d7 shown (n=8 per group). (F) Co-expression of T-bet and Eomes in CD4⁺ T cells over time (representative of at least 2 experiments). (G) Splenic CD4⁺ T cells from naïve mice FACS sorted to 4 subsets based on Il10^{GFP} and Foxp3^{RFP} expression and T-bet and Eomes evaluated (representative of 2 experiments). Data represents mean±SEM.

FIGS. 7A-M are graphical representations showing that Recipient DC and macrophage-derived IL-27 promote the development of T_{R}1 cells. (A-K) B6D2F1 mice were transplanted with TCD BM and CD4⁺ T cells and spleen examined. (A) Correlation of T_{R}1 cells (Il10^{GFP+}Foxp3^{RFPneg}) with proportions of recipient DC at d14 (n=26). (B) Frequencies of T_{reg}(Foxp3GFP+) and T_{R}1 (IFNγ⁺IL-10⁺) cells at d14 in the presence or absence of CD40L inhibition (n=8 per group, grafts were CD4⁺ Foxp3^{GFPneg}). (C) WT.B6D2F1 or CD11c-DOG×DBA/2 F1 recipients were treated with DT to deplete recipient cDC and received B6.WT or MHC-II^{-/-} BM respectively. Expression of T_{R}1, T_{reg} cells, Eomes and IL-10 at d14 are shown (n=10 and 7 respectively). (D) Recipients of WT or CD11c-DOG BM were treated with DT to deplete donor cDC with expression of T_{R}1 and T_{reg} cells at d10 shown (n=10 per group). (E) Data from (A) and (B) demonstrate correlation between numbers of T_{R}1 cells and IL-27⁺ cells per spleen at d14 (n=20). (F) Recipients were treated with IL-6R and spleens analyzed at d5. Phosphorylation of STAT1 and STAT3 in response to IL-6 or IL-27 (n=10 per group). (G and H) Recipients were treated with IL-6R and spleens analyzed at d10. (G) Expression of Foxp3^{RFPneg}I10^{GFP+} T_{R}1, Foxp3^{RFP+} T_{reg}, Eomes and IL-10 in donor CD4⁺ T cells and (H) numbers of IL-27⁺ cells with intensity (MFI) of IL-27 (n=9-10 per group). (I and J) Phenotypes of CD3^{neg} IL-27 secreting cells at d14 are shown. (K) Expression of IL-27 from recipient DC at d+1 after BMT. (L and M) B6.WT or B6.Foxp3^{GFP-DTR} mice were treated with DT for up to 2 weeks and spleens analyzed. (L) Phenotype of IL-27 secreting macrophage in CD3^{neg} splenocytes and (M) expression of Eomes⁺IL-10⁺ cells over time with representative plots at d14. Data represents mean±SEM.

FIGS. 8A-E are graphical representations showing that co-expression of T-bet and Eomes identifies a T_{R}1 cells enriched population in human CD4⁺ T cells. (A) Representative plots show the correlation of Eomes to CD25, FOXP3 and cytokines in CD4⁺ T cells in healthy individuals and at d60 after clinical allo-BMT. (B) Frequencies of T_{R}1 cells defined as IFNγ⁺IL-10⁺ or Eomes⁺IL-10⁺in CD4⁺ T cells in healthy donors (n=27) or d60 after clinical allo-BMT (n=43). (C-E) Expression of cytokines in the T-bet^{lo}Eomes^{hi} population relative to total CD4⁺ T cells or subpopulations defined with differential expression of Eomes and T-bet in healthy individuals (HD, n=27) and at d60 after allo-BMT (BMT, n=43). Data represents median f interquartile range.

FIG. 9 is a graphical representation showing that T_{R}1 cells are suppressive in vitro. Representative plots (of FIG. 1g) show in vitro suppression of proliferation of CFSE labelled B6 CD4⁺ and CD8⁺ responder T cells by naïve T_{reg} cells versus T_{R}1, T_{reg} and T_{con} “suppressors” sorted from transplant recipients at d14 (responder to suppressor at 4:1 ratio).

FIGS. 10A-D are graphical representations showing that T_{R}1 cells display a distinct profile of markers. (A and B) B6D2F1 mice were transplanted with B6 Il10^{GFP}Foxp3^{RFP} CD3⁺ or CD4⁺ T cells and splenic phenotypes examined at d14. (A) Expression of LAG-3/CD49b and FoxP3/IL-10 in CD4⁺ T cell subsets at d14. (B) Representative plots demonstrate the expression of surface molecules in T_{R}1 (FoxP3^{neg}IL-10⁺, red), T_{reg} (FoxP3⁺, blue) and T_{con}(FoxP3^{neg}IL-10^{neg}, green) cells as compared to isotype controls (solid shade). (Data are representative of >2 experiments). (C and D) T_{R}1, T_{reg} and T_{con} cells are processed as described in FIG. 2A. Expression of IL-10 and IFNγ by intracellular cytokine staining (ICS) and expression of Eomes in T cell subsets. Data represents mean f S.E.M.

FIGS. 11A-G are graphical representations showing that Eomes is required for the development of T_{R}1 cells after BMT. (A-F) B6D2F1 recipients were transplanted with B6.WT or Eomes^{-/-} CD4⁺ T cells and spleens examined at d14 as described in FIG. 3A. (A) Absolute numbers of donor CD4⁺ T cells and IFNγ⁺IL-10⁺ T_{R}1 cells (n=10 per group), (B-F) Frequencies and numbers of CD4⁺Gzmb⁺ cells (n=8 per group), IFNγ⁺ TNF⁺ cells (n=9 per group), IL-17A⁺ cells (n=10 per group), IL-4⁺ cells (n=9 per group) and CD4⁺ FoxP3⁺ T_{reg} (n=10 per group). (G) B6.WT or Eomes^{-/-} CD4⁺ T cells were retrovirally transduced with Eomes and transplanted into B6D2F1 recipients. Expression of Gzmb, FoxP3, IL-4 and IL-17A in splenic CD4⁺ T cells were examined at d7 as described in FIG. 3B. Data represents mean f S.E.M.

FIGS. 12A-B are graphical representations showing expression of Eomes during in vitro culture. CD4⁺ T cells were cultured in vitro in polarizing conditions as described in Methods. (A) Expression of Eomes and lineage defining cytokines or transcription factors were determined by FACS on day 4 or day 7 and (B) expression of Eomes in T_{H}1, T_{H}2, T_{H}17 cells relative to T_{R}1 cells quantified with RT-PCR on day 4 (data are from triplicate reactions). Data represents mean f S.E.M.

FIGS. 13A-C are graphical representations showing role of Eomes in the generation of T_{R}1 cells in vitro. (A) Generation of T_{R}1 cells at d6 after culture in WT or gene deficient CD4⁺ T cells. (B and C) Retrovirally transduced (Mock-GFP or Eomes-GFP) CD4⁺ T cells were cultured in the presence of IL-27 as described in the methods. (B) Expression of Eomes and cytokines at d5 after culture (from 3 experiments). (C) Gene expression profiles at d4 of culture quantified by RT-PCR (data are from quadruplicate reactions, representative of 2 experiments). Data represents mean f S.E.M.

FIGS. 14A-F are graphical representations showing that Eomes⁺ T_{R}1 cells are dependent on Blimp-1, IL-27 and IL-10. (A and B) B6D2F1 mice were transplanted with WT or gene deficient CD4⁺ T cells with analysis of spleen at d14. (A) Expression of IL-10 versus Blimp-1 (GFP is driven off the promoter of Prdm1) in CD4⁺ T cells (representative of 2 experiments). (B) Expression of T-bet and Eomes in donor CD4⁺ T cells in recipients of WT or Blimp-1^{-/-} T cells. (C and D) B6D2F1 recipients were transplanted with WT or Blimp-1^{-/-} CD4⁺ T cells that were transduced with empty (Mock-GFP) or Eomes (Eomes-GFP) retrovirus and spleens examined at d7-10 after BMT. (C) Frequencies and number of Granzyme B (n=5 per group) at d7. (D) Frequencies of IL-2 (n=9-10 per group), IL-4 (n=5 per group), IL-17A (n=9-10 per group), GM-CSF (n=9 per group) and FoxP3 (n=18, 17 for WT; n=13, 14 for Blimp-1^{-/-}) with numbers from one representative experiment (n=5 per group). (E and F) B6D2F1 mice were transplanted with WT or gene deficient CD4⁺ T cells with analysis of spleen at d14. (E) Expression of T_{R}1 cells, IFNγ and IL-10 in recipients of WT or Il27r^{-/-} CD4⁺ T cells (n=10 per group). (F) Number of donor CD4⁺ T cells and frequencies of T-bet in recipients of WT or Il10r^{-/-} CD4⁺ T cells (n=10 per group). Data represents mean f S.E.M.

FIG. 15 is a graphical representation showing T_{reg} development in Il27r^{-/-} and Il10^{-/-} T cells after BMT. B6D2F1 recipients were transplanted with WT or gene deficient CD4⁺ T cells and spleens taken at d14. (A) Frequencies and number of T_{reg} cells in recipients of WT or Il27r^{-/-} CD4⁺ T cells (n=10 per group). (B) Frequencies and number of T_{reg} cells in recipients of WT or Il10^{-/-} CD4⁺CD25^{neg} T cells (n=5 per group).

FIGS. 16A-I are graphical representations showing that both T-bet and Eomes are required for T_{R}1 cell generation. (A-D) B6.WT or Tbx21^{-/-} CD4⁺ T cells were transplanted into B6D2F1 mice and spleen examined at d12. (A) Number of donor CD4⁺ T cells and IFNγ⁺IL-10⁺ T_{R}1 cells. (B) Expression of IL-4, IL-17A, IL-10 and IFNγ in CD4⁺ T cells (n=10 per group). (C) Gene expression profile of WT or Tbx21^{-/-} CD4⁺ T cells d12 after BMT (n=4 per group, representative of 2 independent experiments). (D) Expression of IFNγ and Eomes in donor CD4⁺ T cells and of IL-4 and Eomes in CD4⁺IL-10⁺ cells (representative of 4 experiments). (E and F) B6D2F1 mice were transplanted with retrovirally (Mock-GFP and Eomes-GFP) transduced B6.WT or Tbx21^{-/-} CD4⁺ T cells and spleen examined at d7. (E) Expression of IFNγ and Eomes in donor CD4⁺ T cells and (F) frequencies and numbers of CD4⁺IL-17A⁺ T cells. (G) B6 CD4⁺ T cells were transplanted into B6D2F1 recipients and spleens examined. Expression of Eomes in T_{R}1, T_{reg} and T_{con} cells over time (n=7−8 each time-point). (H) B6 Foxp3^{RFP}/Il10^{GFP} CD4⁺ T cells were transplanted into B6D2F1 recipients. Donor CD4⁺ T cells (2×10⁶) were FACS purified from spleens (B6D2F1) d14 days after primary transplant and transplanted into secondary B6D2F1 recipients. Expression of T-bet, Eomes and T_{R}1 cells in CD4⁺ T cells at 4 weeks after transfer into secondary BMT recipients are shown (representative 2 experiments). (I) Expression of T-bet and Eomes in retrovirally transduced CD4⁺ T cells d7-10 after BMT (representative>3 experiments). Data represents mean f S.E.M.

FIG. 17 is a graphical representation showing that recipient DC and donor IL-27 promote T_{R}1 cell development after experimental BMT. (A-D) B6D2F1 mice were transplanted with TCD BM and WT or gene deficient CD4⁺ T cells and spleen examined. (A) B6 Il10^{GFP}Foxp3^{RFP}CD4⁺ T were transplanted into differentially irradiated (1300, 900 or 700 cGy) B6D2F1 mice and frequencies of T_{R}1 cells and recipient DC in spleen determined at d14 (n=7, 11 and 8 respectively). (B) Recipients were transplanted with WT or CD11c-DOG BM and treated with DT after BMT to deplete donor cDC. Expression of cDC and IL-27 in the spleen at d10 is shown (n=10 per group). (C) B6 Il10^{GFP}Foxp3^{RFP} CD4⁺ T and B6.WT or MHC-II^{-/-} BM were transplanted into WT B6D2F1 mice and expression of T_{R}1 cells determined in spleen at d14 (n=5 per group). (D) Recipients were treated with IL-6R and spleens taken for analysis at d10. Number of Foxp3^{RFPneg}Il10^{GFP+}T_{R}1, Foxp3^{RFP+} T_{reg} and Eomes⁺IL-10⁺ cells (n=9-10 per group). Data represents mean f S.E.M.

FIG. 18 is a graphical representation showing that Eomes and T-bet can be used to identify T_{R}1 cells in humans. Expression of memory markers in the T-bet^{lo}Eomes^{hi} population relative to total CD4⁺ T cells in healthy individuals (n=27) or at d60 after clinical BMT (n=43). Data represents median f interquartile range.

FIG. 19 is a schematic representation illustrating a proposed cellular and transcriptional regulation of T_{R}1 cell development after BMT.

FIG. 20 is a graphical representation showing that Eomes over-expressing T_{R}1 cells are independent of IL-27. B6 splenic CD4⁺ T cells were retrovirally transduced with Eomes and then polarized to T_{R}1 cells after stimulation with plate bound CD3 and CD28. Representative plots show the frequency of IL-10⁺IFNγ⁺ T_{R}1 cells generated in the absence or presence of IL-27.

FIG. 21 is a graphical representation showing introduction of Eomes into alloantigen specific CD4⁺ T cells. B6 splenic CD4⁺ T cells were transduced in allo-antigen nonspecific (stimulated with CD3/CD28) or allo-antigen specific (stimulated with allo-DC) manners respectively. Representative FACS plots showed the expression of GFP (indicating successful retroviral transduction) 2-4 days after retroviral transduction.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

As used herein, the term “anergy” or “tolerance” refers to insensitivity of T cells to T cell receptor-mediated stimulation. Such insensitivity is generally antigen-specific and persists after exposure to the tolerizing antigen has ceased. For example, anergy in T cells (as opposed to unresponsiveness) is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. 1992 Science. 257:1134).

“Autoimmunity” refers to persistent and progressive immune reactions to non-infectious self-antigens, as distinct from infectious non-self-antigens from bacterial, viral, fungal, or parasitic organisms which invade and persist within mammals and humans. Autoimmune conditions include scleroderma, Grave's disease, Crohn's disease, Sjogren's disease, multiple sclerosis, Hashimoto's disease, psoriasis, myasthenia gravis, Autoimmune Polyendocrinopathy syndromes, Type I diabetes mellitus (TIDM), autoimmune gastritis, autoimmune uveoretinitis, polymyositis, colitis, and thyroiditis, as well as in the generalized autoimmune diseases typified by human Lupus. “Autoantigen” or “self-antigen” as used herein refers to an antigen or epitope which is native to the mammal and which is immunogenic in said mammal disease. A patient with an autoimmune disease may be diagnosed as known to one of ordinary skill in the art. Such patients may be identified symptomatically and/or by obtaining a sample from a patient and isolating autoreactive T cells and comparing the level of autoreactive T cells in a patient to a control (see, U.S. Patent Application Publication No. 20060105336). For instance, type 1 diabetes may be identified by age of on-set and dependence on insulin injections to maintain glucose homeostasis. The response of a patient with an autoimmune disease to treatment may be monitored by determining the severity of their symptoms or by determining the frequency of autoreactive T cells in a sample from a patient with an autoimmune disease. The severity of symptoms of the autoimmune disease may correlate with the number of autoreactive T cells (see, U.S. Patent Application Publication No. 20060105336). In addition, an increase in the number of autoreactive T cells in the sample may be used as an indication to apply treatments intended to minimize the severity of the symptoms and/or treat the disease before the symptoms appear.

By “coding sequence” is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

The terms “eliciting immune tolerance” and “inducing immune tolerance” are used interchangeably herein to refer to any mechanism by which a potentially injurious immune response is prevented, suppressed, or shifted to a non-injurious immune response, including initiating, triggering, causing, enhancing, amplifying, improving, augmenting or prolonging a state of complete or partial unresponsiveness of the immune system to substances or tissues that have the capacity to elicit an immune response. The initiation or enhancement of immune tolerance can be assessed using assays known to those skilled in the art including, but not limited to, antibody assays (for example ELISA assays), antigen specific cytotoxicity assays and the production of cytokines (for example ELISPOT assays).

Reference to “enriching” should be understood as a reference to increasing the ratio of cells expressing the desired phenotype relative to the cells not expressing the desired phenotype in the starting sample. This is achieved by removing or otherwise reducing the number of cells that do not express the desired phenotype. It should be understood that reference to enrichment is not limited to an enrichment step that removes all the cells not expressing the desired phenotype from the sample. Rather, it is a reference to decreasing the concentration of these suitably undesired cells in the test sample. The decrease in concentration may therefore be of varying degrees. The method of the present invention should be understood to encompass conducting one or more repeated sequential enrichment steps in order to improve the purity of the desired population (such as by performing two or more sequential enrichment steps for any one or more of CD4⁺, CD122⁺, α4β7⁺, LAG-3⁺, Ly6C⁺, and TIGIT⁺, CD25^{lo/}, CD69^{lo/-}, and FoxP3^{lo/-}). The decision as to whether one or more enrichment steps are required to be performed at any given stage can be made by a person skilled in the art on a case by case basis. When T cell numbers are relatively high (such as in a PBMC sample), a single enrichment step may be sufficient to enrich for the desired population. However, where a sample with very low numbers of T cells is used, it may be desirable to perform two or more of each enrichment step in order to maximize the purity of the desired cellular population. For example, in some preferred embodiments, enriching a cell population refers to increasing the percentage by about 10%, by about 20%, by about 30%, by about 40%, by about 50% or greater than 50% of one type of cell in a population of cells as compared to the starting population of cells. In other preferred embodiments, enriched cell populations of the present invention will comprise at least 30%, 40%, 50%, 60% 70%, 80%, 85%, 90%, 95%, 98%, or 99% of the selected cell type. In yet other embodiments, an enriched preparation of immunoregulatory T cells may be described as comprising about 1% or greater or about 0.5% to about 40% of the total cell population contained in a preparation. In some embodiments, the enriched preparations comprise a 100-fold, 200-fold, 500-fold, 1,000-fold, or up to a 2,000-fold or 10,000-fold to 20,000-fold enriched preparation of immunoregulatory T cells. In specific embodiments, enriched T-cell samples refer to those samples or biological samples that have been enriched for T cells by positive selection of the T cells bearing the CD4 marker by determining the levels of expression of the CD4 marker. Other enriched T-cell samples have been enriched for T-cells by negative selection of (i.e., selecting against) non-T-cells which can be distinguished by their levels of expression of other common determinants.

“Eomes” refers to Eomesodermin, a protein that in humans is encoded by the Eomes gene. Eomes is a transcriptional regulator. Representative coding sequences forum human Eomes are set forth in NCBI Accessions NM_001278182, NM_005442 and NM_001278183.

The term “exogenous” refers to any material introduced from or produced outside an organism, cell, tissue or system.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

As used herein, the term “graft” or “transplant” refers to an organ, tissue, or cell that has been transplanted from one subject to a different subject, or transplanted within the same subject (e.g., to a different area within the subject). Organs such as liver, kidney, heart or lung, or other body parts, such as bone or skeletal matrix such as bone marrow, tissue, such as skin, intestines, endocrine glands, or stem cells of various types, or hematopoietic cells including hematopoietic stem and progenitor cells, are all examples of transplants. The graft or transplant can be an allograft, autograft, isograft or xenograft. The term “allograft” refers to a graft between two genetically non-identical members of a species. The term “autograft” refers to a graft from one area to another on a single individual. The term “isograft” or “syngraft” refers to a graft between two genetically identical individuals. The term “xenograft” refers to a graft between members of different species.

Immune conditions, diseases, disorders and reactions or responses to be treated according to the methods and compositions of the invention means a disease in which the immune system contributes to pathogenesis. These reactions include, but are not limited to, inflammatory disorders, cancer, autoimmune conditions, disorders or diseases and persistent and progressive immune reactions to infectious non-self-antigens from bacterial, viral (e.g., HCV), fungal, or parasitic organisms which invade and persist within mammals and humans. Such conditions and disorders include allergies and/or asthma. The allergies and asthma may be due to sensitization with foreign or non-self-antigens as pollen, animal dander and food proteins. The source of the provoking foreign antigen can be plant, fungal, mold, or other environmental contaminants.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly brought about by T cell activation, e.g., antibody production (humoral responses) and activation of cytokine responsive cells, e.g., macrophages. Immune cells involved in the immune response include lymphocytes, such as B cells and T cells (CD4⁺, CD8⁺, Th1 and Th2 cells); antigen presenting cells (e.g., professional antigen presenting cells such as B lymphocytes, monocytes, dendritic cells, Langerhans cells, and non-professional antigen presenting cells such as keratinocytes, endothelial cells, astrocytes, fibroblasts, oligodendrocytes); natural killer cells; myeloid cells, such as monocytes, macrophages, eosinophils, mast cells, basophils, and granulocytes.

The term “immunoregulatory” refers to an agent that inhibits or reduces one or more biological activities of the immune system. An immunoregulatory agent is an agent that inhibits or reduces one or more biological activities (e.g., the proliferation, differentiation, priming, effector function, production of cytokines or expression of antigens) of one or more immune cells (e.g., T cells).

The term “isolated cell” as used herein refers to a cell that has been removed from an organism in which it was originally found, or a descendant of such a cell. Optionally, the cell has been cultured in vitro, e.g., in the presence of other cells. Optionally, the cell has been cultured in vitro, e.g., in the presence of other cells.

The term “isolated population” with respect to an isolated population of cells as used herein refers to a population of cells that has been removed and separated from a mixed or heterogenous population of cells. In some embodiments, an isolated population is a substantially homogenous population of cells as compared to the heterogenous population from which the cells were isolated or enriched. In some embodiments, the isolated population is an isolated population of immunoregulatory T cells, e.g., a substantially homogenous population of human immunoregulatory T cells as compared to a heterogenous population of cells comprising immunoregulatory T cells from which the human immunoregulatory T cells were derived. Isolated populations will typically comprise a plurality of cells, preferably at least 10³, 10⁴, 10⁵, 10⁶, 10⁷, 10⁸, 10⁹, 10¹⁰, or 10¹¹ cells. The population in some embodiments has from 10⁵ to 10⁷ cells, 10⁶ to 10⁸ cells, or from 10⁸ to 10¹¹ cells, or 10¹⁰ to 10¹² cells.

A “marker” and “cell marker” and the like, as used herein in the context of a cell, means any trait or characteristic in the form of a chemical or biological entity (including phenotypic and genotypic traits) that is identifiably associated with, or specifically found in or on a particular cell, cell population or tissue, including those identified in or on a tissue or cell population affected by a disease or disorder. Markers may be morphological, functional or biochemical in nature and may be genotypic or phenotypic. In preferred embodiments that marker is a cell surface antigen or genetic component that is differentially or preferentially expressed (or is not) by specific cell types (e.g., immunoregulatory T cells) or by cells under certain conditions (e.g., during specific points of the cell cycle or cells in a particular niche). In still other preferred embodiments the marker may comprise a gene or genetic entity that is differentially regulated (up or down) in a specific cell or discrete cell population, a gene that is differentially modified with regard to its physical structure and chemical composition or a protein or collection of proteins physically associated with a gene that show differential chemical modifications. Markers contemplated herein are specifically held to be positive or negative and may denote a cell or cell population by its present (positive) or absence (negative).

By “obtained from” is meant that a sample such as, for example, a cell or tissue sample is isolated from, or derived from, a particular source.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence.

The terms “patient”, “subject”, “host” or “individual” used interchangeably herein, refer to any subject, particularly a vertebrate subject, and even more particularly a mammalian subject, for whom therapy, or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting immune tolerance. However, it will be understood that the aforementioned terms do not imply that symptoms are present.

By “pharmaceutically acceptable carrier” is meant a solid or liquid filler, diluent or encapsulating substance that can be safely used in topical or systemic administration to an animal, preferably a mammal, including humans. Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

A “pharmaceutical composition” is intended to include the combination of an active agent with a carrier, inert or active such as biocompatible scaffold or matrix, making the composition suitable for diagnostic or therapeutic use in vitro, in vivo, or ex vivo.

Reference to “phenotypic profile” should be understood as a reference to the presence or absence of the transcription of the genes encoding the subject markers and/or the cell surface expression of the expression product translated therefrom. It should be appreciated that although most cells falling within the scope of the claimed immunoregulatory T cell populations will be characterized by the presence or absence of the subject marker as a cell surface anchored expression product, some cells falling within the defined populations may initially exhibit changes only at the transcriptome level, such as when the transcription of a given marker has been upregulated but may not yet have resulted in a cell surface anchored expression product. In general, cells which progress to a new differentiative stage will transiently exhibit gene expression changes which are not yet evident in the context of changes to levels of an expression product. However, these cells nevertheless fall within the scope of the claimed cellular populations, although they will not be isolatable by the method defined herein until such time as cell surface marker expression occurs.

“Positive,” “low” and “negative” expression levels as they apply to markers are defined as follows. Cells with negative expression (i.e., “-”) are herein defined as those cells expressing less than, or equal to, the 95^{th} percentile of expression observed with an isotype control antibody in the channel of fluorescence in the present of the complete antibody staining cocktail labelling for other proteins of interest in additional channels of fluorescence emission. Those skilled in the art will appreciate that this procedure for defining negative events is referred to as “fluorescence minus one,” or “FMO,” staining. Cells with expression greater than the 95^{th} percentile of expression observed with an isotype control antibody using the FMO staining procedure described above are herein defined as “positive” (i.e., “+”). As defined herein there are various populations of cells broadly defined as “positive.” First, cells with low expression (i.e., “lo”) age generally defined as those cells with observed expression above the 95^{th} percentile determined using FMO staining with an isotype control antibody and within one standard deviation of the 95^{th} percentile of expression observed with an isotype control antibody using the FMO staining procedure described above. The term “lo” in relation to Tbet^{lo} refers to a distinct cell or population of cells that expresses Tbet at a lower level than one or more other distinct cells or populations of cells.

The term “recombinant expression” and its grammatical equivalents (e.g., “recombinantly expressing”), as used herein, relates to transcription and translation of an exogenous gene in a host cell. Exogenous DNA refers to any deoxyribonucleic acid that originates outside of the host cell. The exogenous DNA may be integrated in the genome of the host cell or may be expressed from a non-integrating element.

“Regulatory sequences”, “regulatory elements”, control elements”, “control sequences” and the like are used interchangeably herein to refer to nucleotide sequences located upstream (5′ non-coding sequences), within, or downstream (3′ non-coding sequences) of a coding sequence, and which influence the transcription, RNA processing or stability, or translation of the associated coding sequence, either directly or indirectly. Regulatory elements include enhancers, promoters, translation leader sequences, introns, Rep recognition element, intergenic regions and polyadenylation signal sequences. They include natural and synthetic sequences as well as sequences which may be a combination of synthetic and natural sequences.

For the purposes of the present invention, the terms “purifying,” “sorting,” “selecting” or “isolating” specific cells, cell populations, or cell populations, may be used interchangeably and mean, unless otherwise dictated by context, that a selected cell or defined subset of cells are removed from a tissue sample or cellular preparation, and separated from other cells and contaminants that are not within the parameters defining the cell or cell population. In some embodiments, an isolated immunoregulatory population of T cells is substantially free from contamination by other cell types. However, when the process or treatment results in a cell population it is understood that it is impractical to provide compositions of absolute purity. In such cases that cell population is “enriched” for the selected cells that then exist in the presence of various contaminants (including other cell types) that do not materially interfere with the function or properties of the selected cell population.

The term “separation” or “selection” as used herein refers to isolating different cell types into one or more populations and collecting the isolated population as a target cell population which is enriched in a specific immunoregulatory T cell population. Selection can be performed using positive selection, whereby a target enriched cell population is retained, or negative selection, whereby non-target cell types are discarded (thereby enriching for desired target cell types in the remaining cell population).

The term “positive selection” as used herein refers to selection of a desired cell type by retaining the cells of interest. In some embodiments, positive selection involves the use of an agent to assist in retaining the cells of interest (e.g., use of a positive selection agent such as an antibody which has specific binding affinity for a surface antigen on the desired or target cell. In some embodiments, positive selection can occur in the absence of a positive selection agent (e.g., in a “touch-free” or closed system), for example, where positive selection of a target cell type is based on any of cell size, density, and/or morphology of the target cell type.

The term “negative selection” as used herein refers to selection of undesired or non-target system cells for depletion of discarding, thereby retaining (and thus enriching) the desired target cell type. In some embodiments, negative selection involves the use of an agent to assist in selecting undesirable cells for discarding, e.g., use of a negative selection agent such as an antibody which has specific binding affinity for a surface antigen on unwanted or non-target cells. In some embodiments, negative selection does not involve a negative selection agent. In some embodiments, negative selection can occur in the absence of a negative selection agent, e.g., in a “touch-free” or closed system, for example, where negative selection of an undesired cell (non-target) cell type.

As used herein, the term “sample” or “biological sample” refers to tissues or body fluids removed from a mammal, preferably human, and which contain immunoregulatory T cells, including, but not limited to, Eomes⁺IL-10⁺ T cells. In some embodiments, the samples are taken from individuals with an immune response which needs to be suppressed. In some embodiments, the individual has an allergy, Graft vs. Host Disease, an organ transplant, or autoimmune disorder. Samples preferably are blood and blood fractions, including peripheral blood. The biological sample is drawn from the body of a mammal, such as a human, and may be blood, bone marrow cells, or similar tissues or cells from an organ afflicted with the unwanted immune response. Methods for obtaining such samples are well known to workers in the fields of cellular immunology and surgery. They include sampling blood in well-known ways or obtaining biopsies from the bone marrow or other tissue or organ. In preferred embodiments, the sample is a T-cell enriched sample in which the sample cells are substantially T-cells.

“Substantially homogeneous” cell population describes a population of cells in which more than about 75%, or alternatively more than 80%, or alternatively more than 85%, or alternatively more than 90%, or alternatively, more than 95%, of the cells are of the same or similar phenotype. Phenotype is determined by the cell surface markers described in more detail herein.

The terms “treating” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, bur has not yet been diagnosed as having it; inhibiting a disorder (i.e., arresting its development); and/or relieving or ameliorating the symptoms of disorder. As it understood by those skilled in the art “treatment” can include systemic amelioration of the symptoms associated with the pathology and/or a delay in onset of symptoms. Clinical and sub-clinical evidence of “treatment” will vary with the pathology, the individual and the treatment.

“Tolerogenic” means silencing or down-modulating an immune response. The term “tolerogenic” also refers to a phenotype of a cell or a substance that induces immune tolerance, typically to an antigen directly or indirectly.

As used herein, underscoring or italicizing the name of a gene shall indicate the gene, in contrast to its protein product, which is indicated by the name of the gene in the absence of any underscoring or italicizing. For example, “Eomes” shall mean the Eomes gene or Eomes polynucleotides, whereas “Eomes” shall indicate the protein product or products generated from transcription and translation and alternative splicing of the “Eomes” gene.

Each embodiment described herein is to be applied mutatis mutandis to each and every embodiment unless specifically stated otherwise.

In order that the invention may be readily understood and put into practical effect, particular preferred embodiments will now be described by way of the following non-limiting examples.

### Experimental 1

### T_{R}1 Cells Represent a Major Regulatory T Cell Population in GVHD

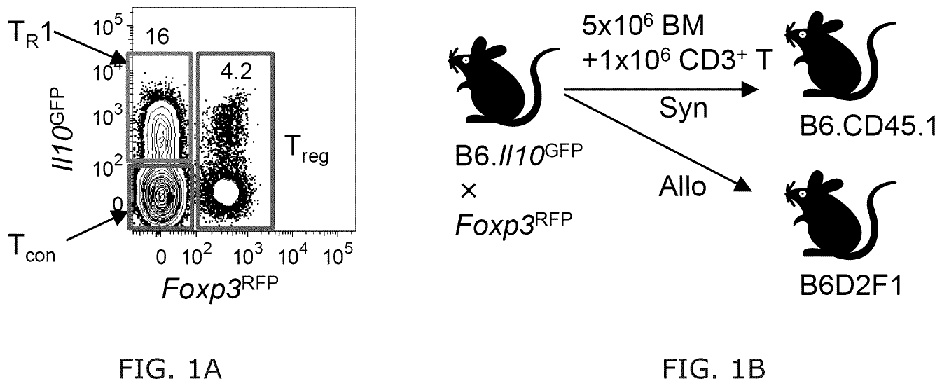

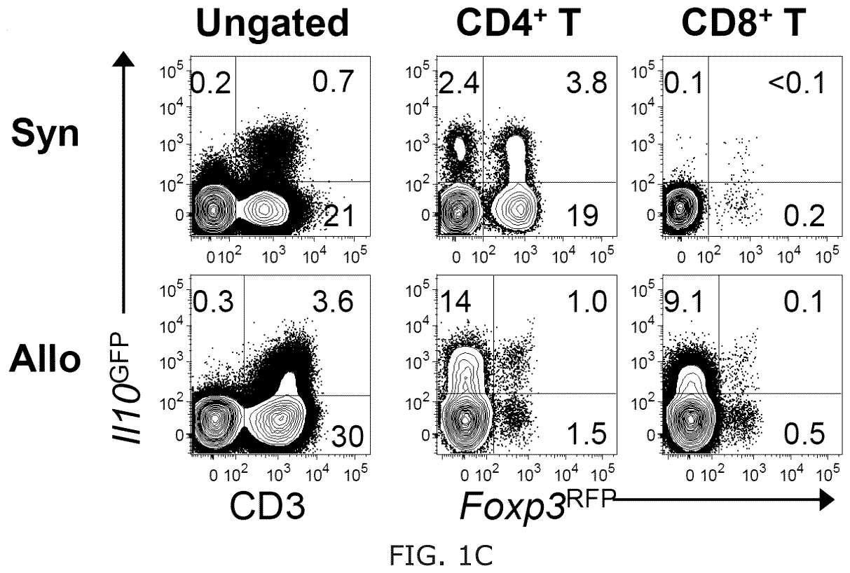

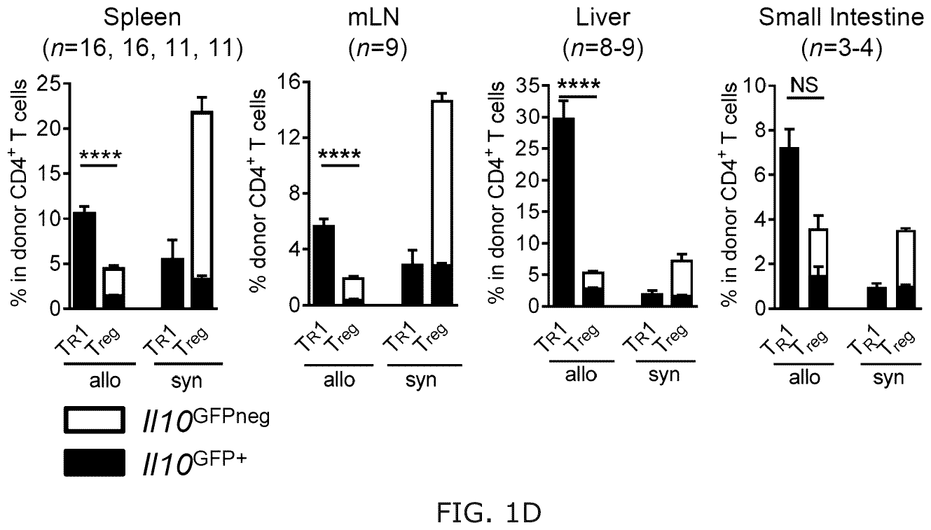

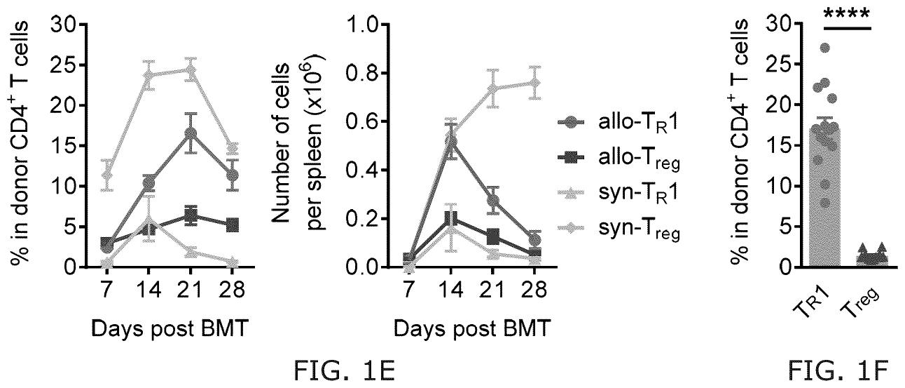

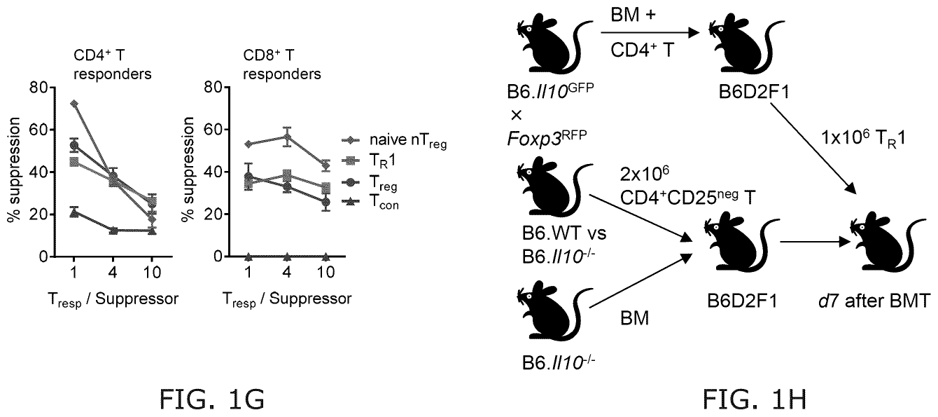

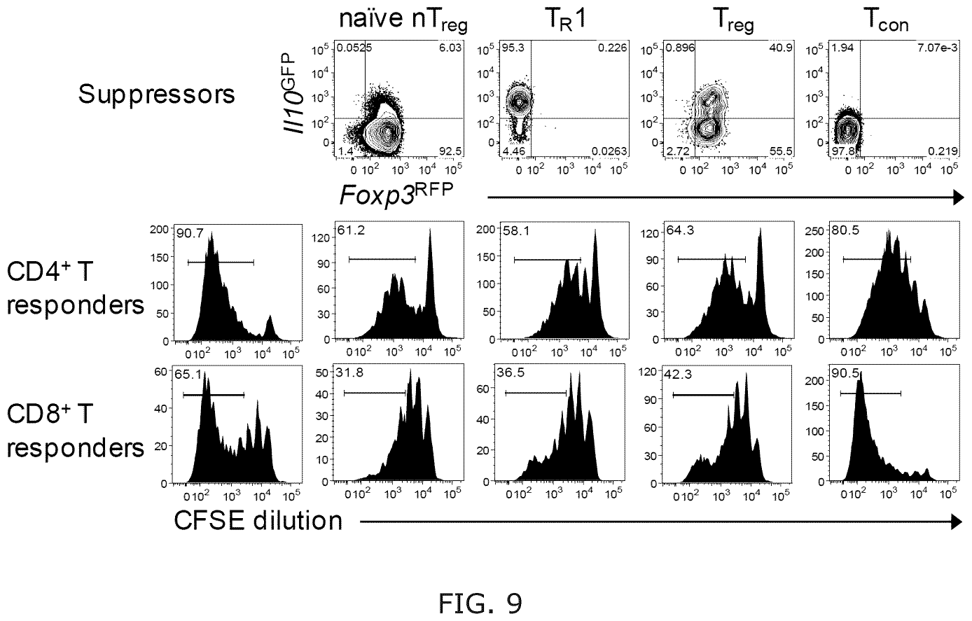

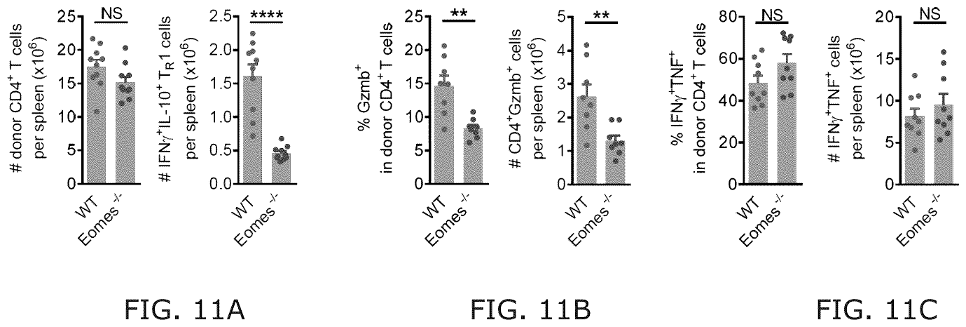

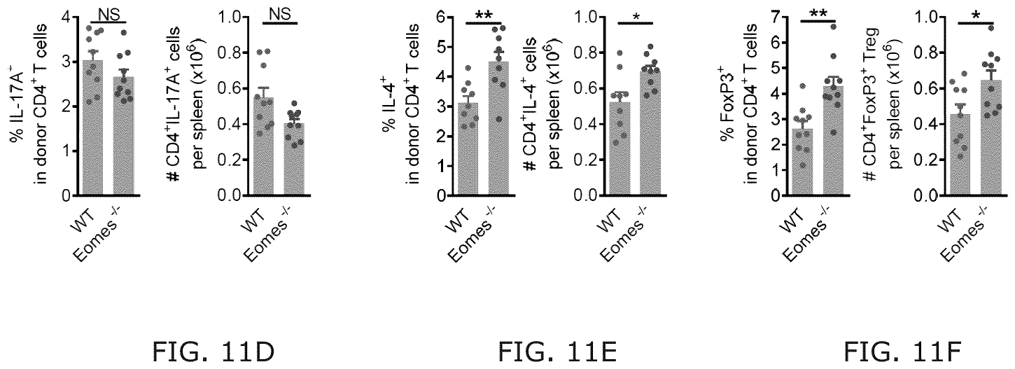

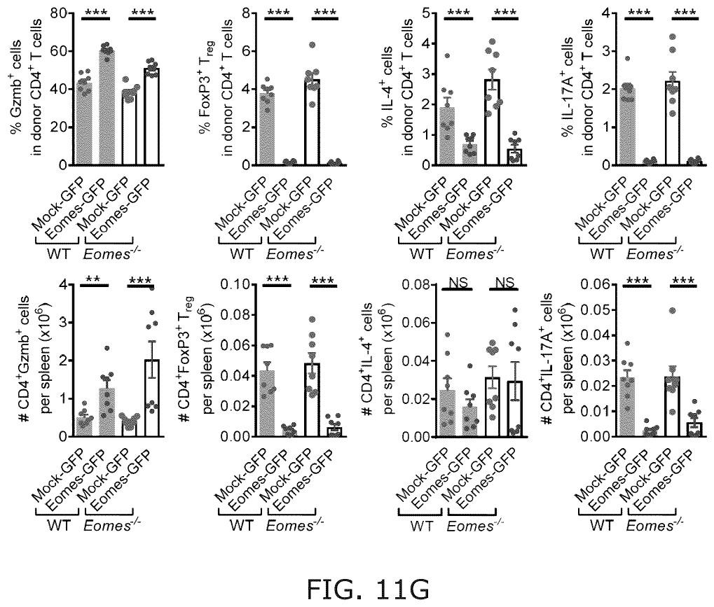

The present inventors used Il10^{GFP} and Foxp3^{RFP} dual reporter mice as BMT donors to define CD4⁺ FoxP3^{neg} IL-10⁺ type-1 regulatory T (T_{R}1) cells, CD4⁺ FoxP3⁺ regulatory T (T_{reg}) cells and CD4⁺ FoxP3^{neg} IL-10^{neg} conventional T (Tcom) cells (FIG. 1A). T cells were the major IL-10 producers after both allogeneic and syngeneic BMT (FIG. 1B), with the highest proportion and intensity of IL-10 produced by T_{R}1 cells (FIG. 1C). Importantly, T_{R}1 cells were present at up to 10-fold higher frequency and number than T_{reg} cells after allogeneic BMT in GVHD target tissues (liver, and to lesser extent small intestine), mesenteric lymph nodes (FIG. 1D) and spleen (FIG. 1D-F). T_{R}1 cells induced under these conditions had suppressive properties in vitro equivalent to post-transplant T_{reg} cells on a per cell basis (FIGS. 1G, 9). To confirm their suppressive function in vivo, GVHD was induced with WT or Il10^{-/-} CD4⁺CD25^{neg} T cells that cannot develop into functional T_{R}1 cells. As expected, enhanced GVHD was observed in the absence of IL-10; however, adoptive transfer of limited numbers of T_{R}1 cells at d7 after BMT (FIG. 1H) when acute GVHD was established, prolonged survival significantly (FIG. 11), consistent with potent regulatory function. Thus, T_{R}1 cells represent the major regulatory T cell population in GVHD induced by allogeneic BMT and contribute significantly to transplant survival.

### T_{R}1 Cells Express Eomes and Display a Distinct Phenotypic Profile

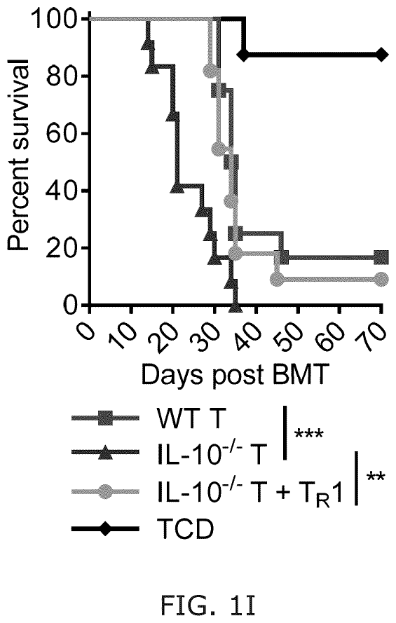

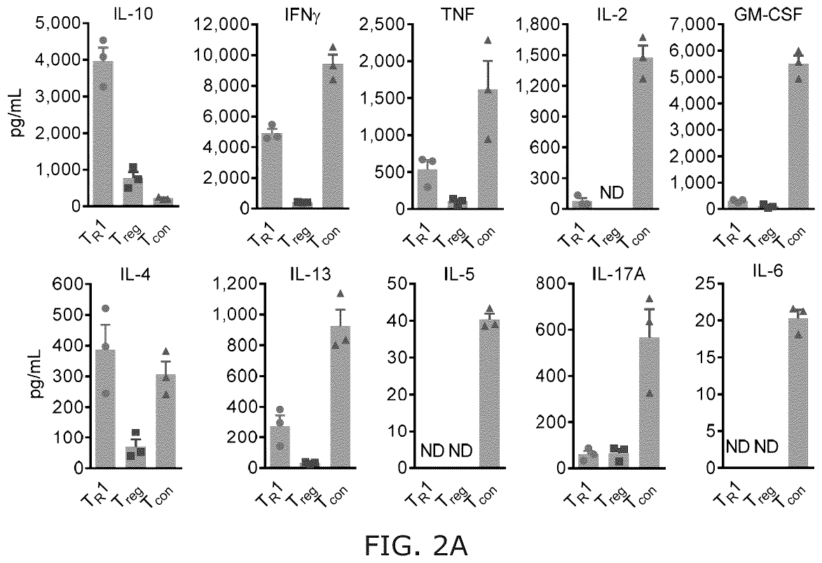

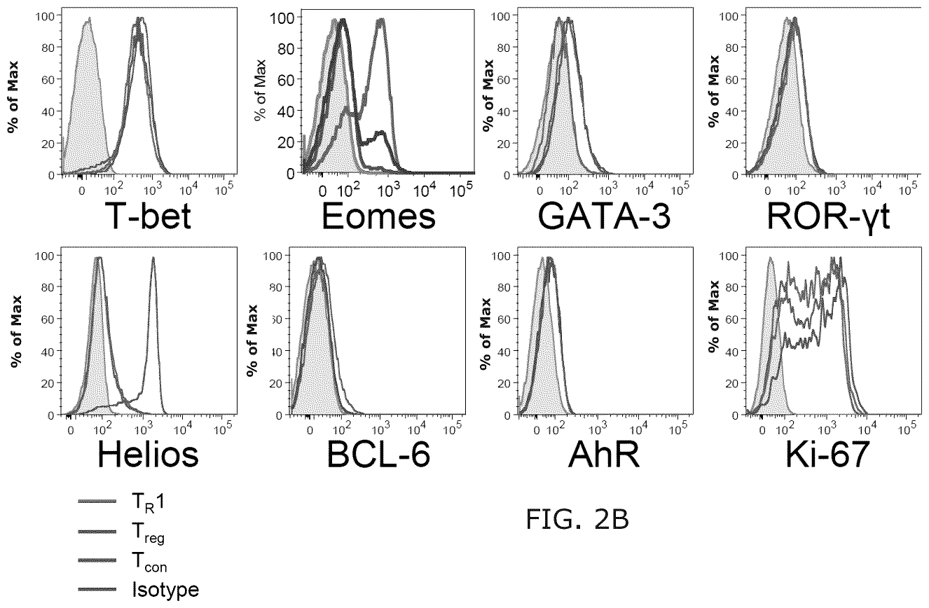

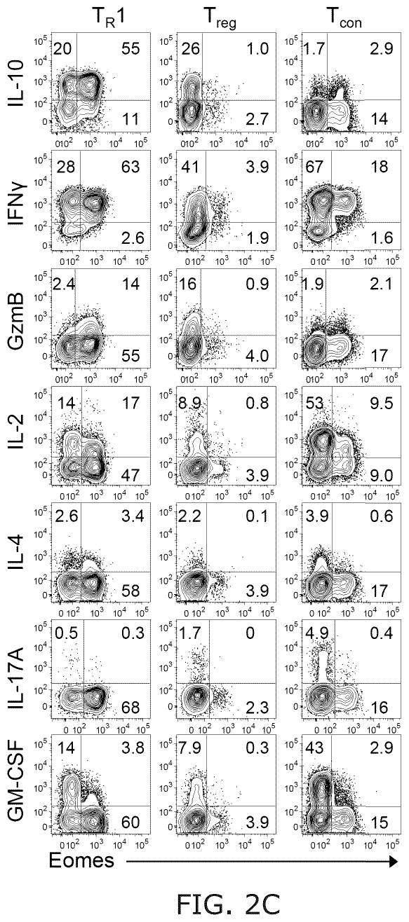

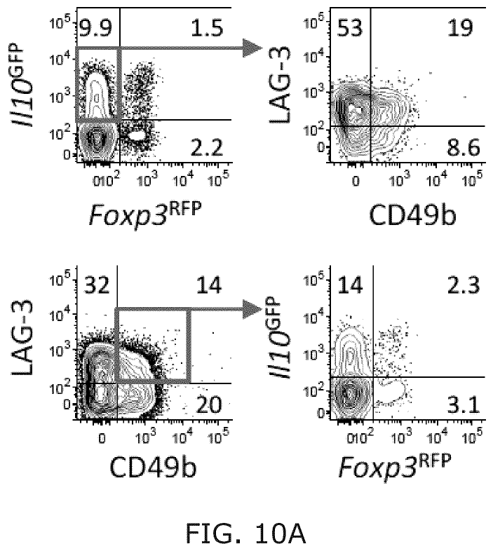

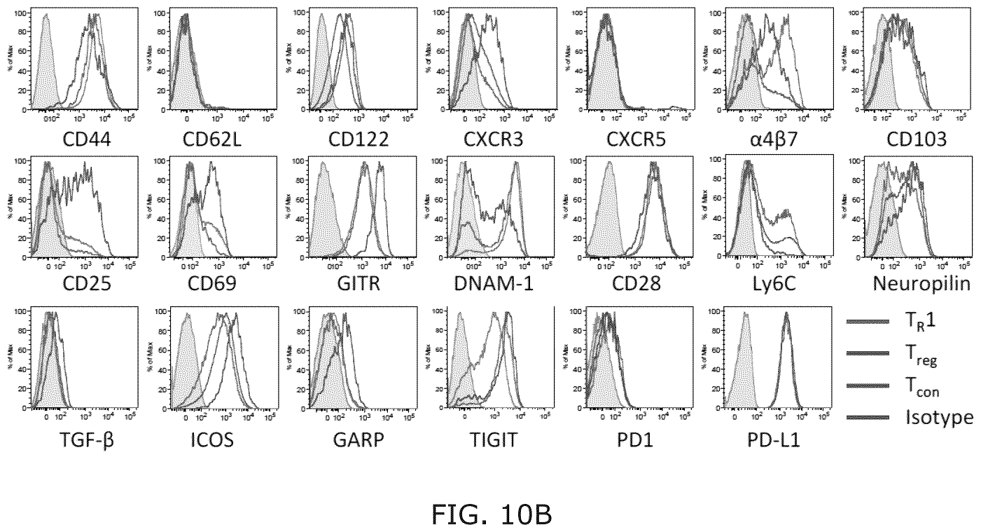

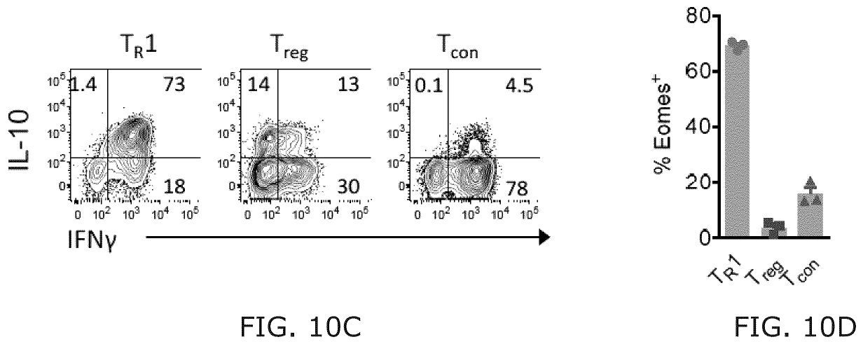

CD49b and LAG-3 co-expression can be used to identify T_{R}1 cells in models of colitis (9), however, their expression is insufficient to identify T_{R}1 cells after BMT (FIG. 10A). The present inventors therefore used Foxp3^{RFPneg} and Il10^{GFP+} as T_{R}1 cell markers. Thus defined T_{R}1 cells demonstrated high expression of CD122, α4β7, LAG-3, Ly6C and TIGIT, and low expression of CD25 and CD69 relative to other CD4⁺ T cell subsets (FIG. 10B). Consistent with the T_{R}1 cell phenotype (3, 5, 9), Foxp3^{RFPneg}Il10^{GFP+} T_{R}1 cells expressed high amounts of IL-10 and IFNγ but little T_{H}2 cytokines such as IL-4, IL-13 and IL-5, or T_{H}17 cytokines such as IL-17, IL-6 or GM-CSF (FIGS. 2A, 10C).

T_{R}1 cells have often been considered a terminally differentiated T_{H}1 cell subset programmed to limit aberrant inflammation (5, 13, 22). Indeed, T_{R}1 cells expressed high amounts of T-bet, the T_{H}1 determining transcription factor, but low amounts of GATA-3, BCL-6 and ROR-γt. Strikingly, when the present inventors analysed the expression of other transcription factors related to T cell differentiation, high Eomes expression was observed, which was largely restricted to T_{R}1 cells (FIGS. 2B, 10D). Eomes expression tightly correlated with high expression of IL-10, IFNγ and granzyme B (GzmB) (FIG. 2C). In contrast, Eomes⁺ T_{R}1 cells expressed low levels of IL-2, IL-17A and GM-CSF (FIG. 2C). Thus, T_{R}1 cells that develop during allogeneic BMT specifically express Eomes.

### Eomes is Required for T_{R}1 Cell Differentiation

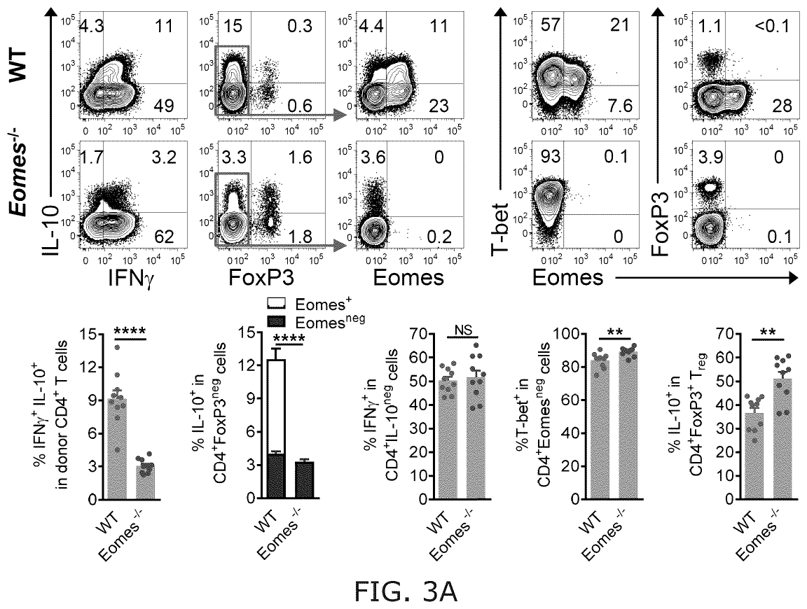

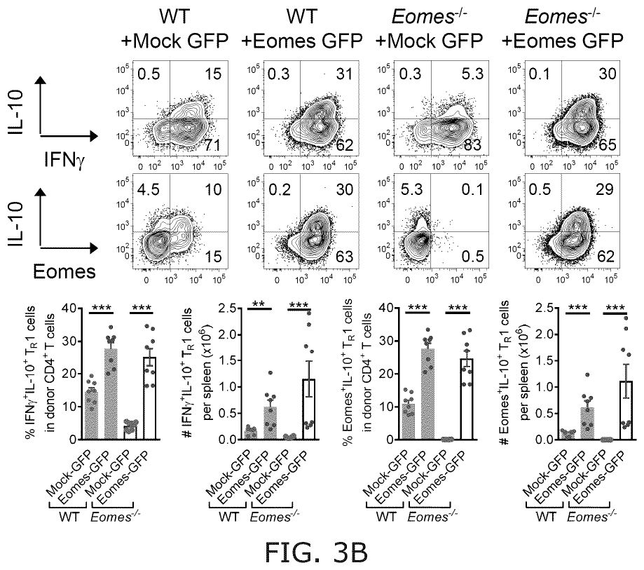

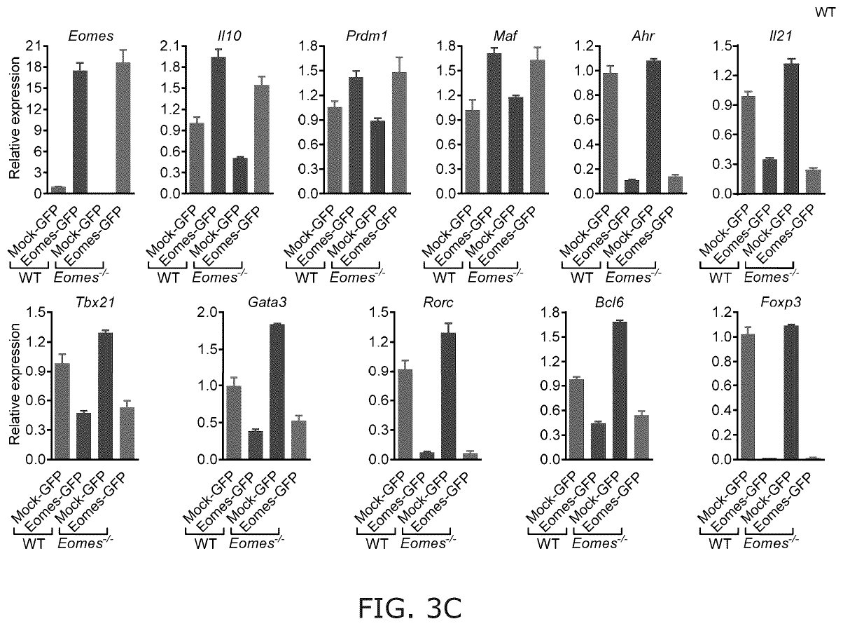

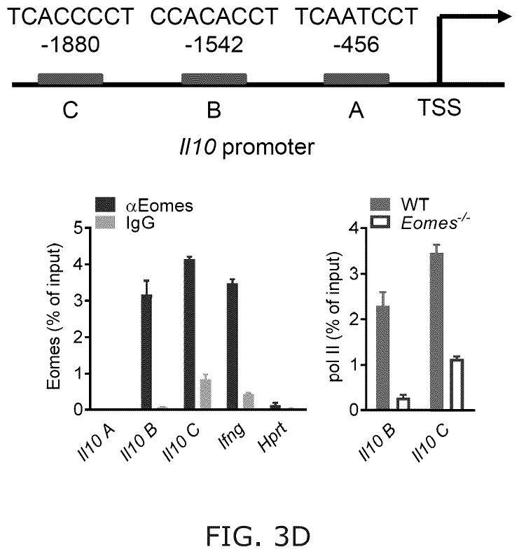

To test the role of Eomes in T_{R}1 cell development in vivo, the present inventors used CD4⁺ T cells isolated from Eomes^{fl/fl}xCd4-cre donor mice in allogeneic BMT. Strikingly, T_{R}1 cell generation was significantly reduced (by >70%) with decreased Gzmb expression in recipients of Eomes-deficient CD4⁺ T cells (FIGS. 3A, 11A,B). Critically, the loss of Eomes did not impair the development of IL-10^{neg}IFNγ⁺ or T-bet⁺ T_{con}, IFNγ⁺ TNF⁺T_{H}1, IL-17A⁺ T_{H}17 cells or IL-10 expression by T_{reg} cells but instead favoured the expression of IL-4 and FoxP3 (FIGS. 3A, 11B-F). To further elucidate the role of Eomes in the differentiation of T_{R}1 cells and transactivation of Il10, the present inventors transplanted donor WT or Eomes^{-/-} CD4⁺ T cells which constitutively expressed Eomes after retroviral transduction. Strikingly, enforced expression of Eomes rescued the development of T_{R}1 cells from Eomes^{-/-} CD4⁺ T cells after BMT and also promoted their development in WT cells (FIG. 3B). In addition, over-expression of Eomes promoted the expression of Gzmb whilst suppressing FoxP3, IL-4 and IL-17A expression (FIG. 11G). Furthermore, over-expression of Eomes upregulated the transcription of Il10 but suppressed that of other lineage defining transcription factors including Tbx21, Gata3, Rorc, BcI6 and Foxp3 in addition to the T_{R}1/T_{H}17 related factors Ahr and Il21 (10, 23, 24)(FIG. 3C).

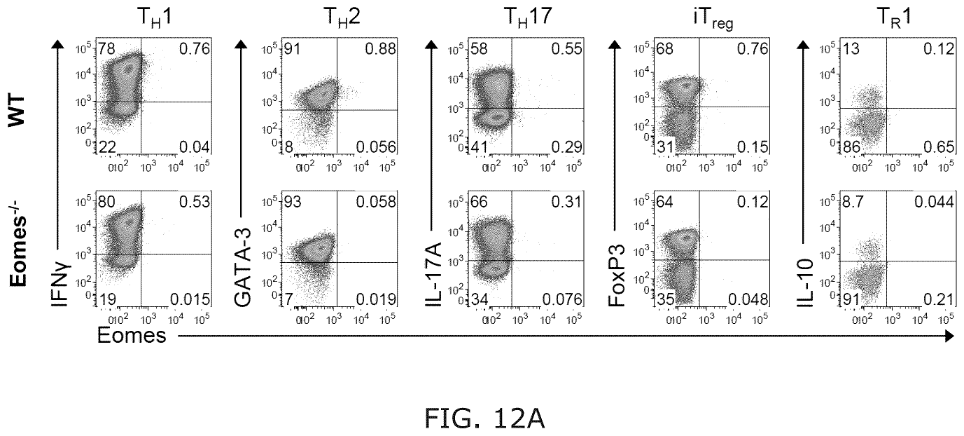

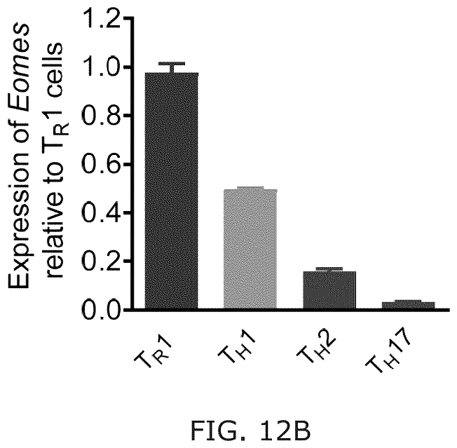

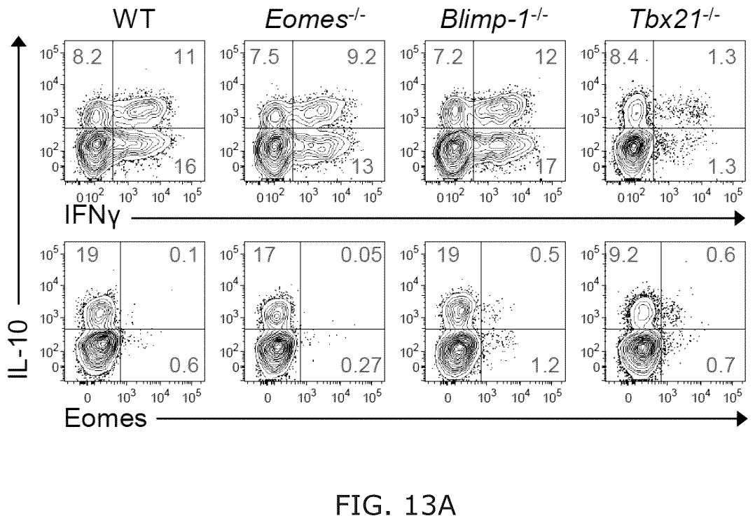

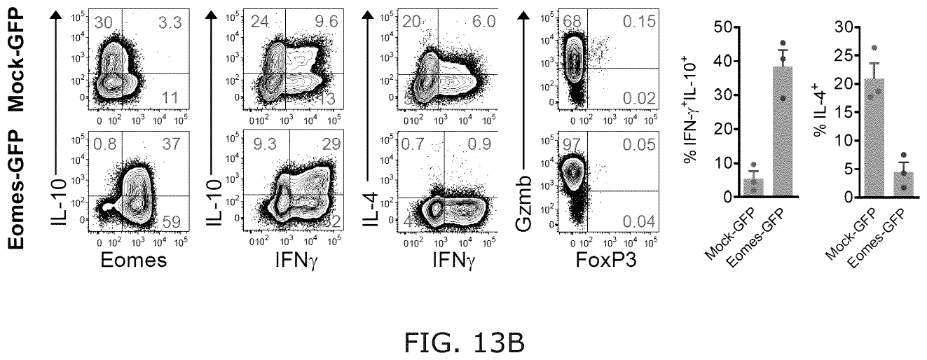

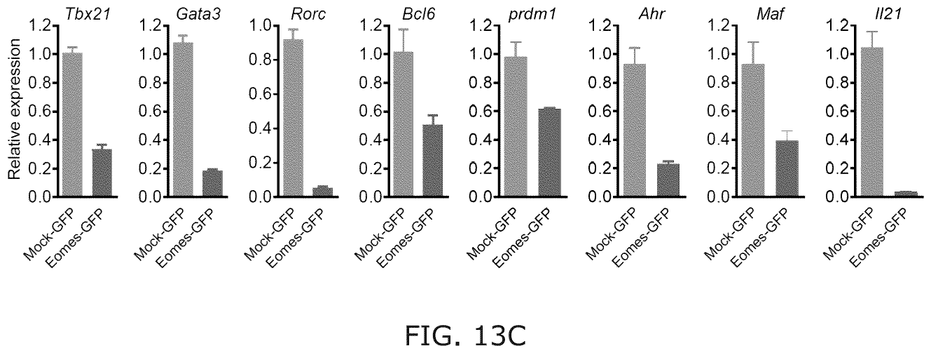

Notably, T_{R}1 cells generated in vitro in the presence of IL-27, a cytokine promoting T_{R}1 cell development (8, 11, 12), did not express Eomes protein, nor did T_{H}1, T_{H}2, T_{H}17, iT_{reg} cells (FIG. 12A), indicating that short-term in vitro cultures do not replicate the conditions inducing T_{R}1 cells after BMT. Nevertheless, Eomes mRNA was higher in T_{R}1 than other T cell lineages in these cultures (FIG. 12B). Consistent with this observation, a defect in T_{R}1 differentiation was not observed in the absence of Eomes in these conditions (FIG. 13A). However, transduction of Eomes into CD4⁺ T cells and subsequent re-stimulation in culture dramatically promoted the differentiation of IL-10⁺IFNγ⁺ T_{R}1 cells and the expression of granzyme B, while suppressing the expression of IL-4 and FoxP3 (FIG. 13B). Over-expression of Eomes also suppressed mRNA expression of transcription factors defining other T_{H} lineages, including Tbx21, Gata3, Rorc and BcI6 and other T_{R}1/T_{H}17 related factors, like Ahr, Maf and I/21(FIG. 13C). Collectively, the present inventors show that Eomes is required for T_{R}1 differentiation and IL-10 secretion and repression of alternative fate differentiation

### EOMES Directly Regulates IL-10 Expression in T_{R}1 Cells

To understand the mechanism by which Eomes regulates T_{R}1 cell differentiation, chromatin immunoprecipitation (ChIP) assays was performed on sort purified T_{R}1 cells or CD4⁺ T cells 14 days after BMT. This demonstrated that Eomes is bound to multiple sites within 2 kb upstream of the transcription start site (TSS) of the Il10 gene (FIG. 3D). The binding of Eomes to the Il10 promoter was similar to that observed in the Ifnγ promoter, suggesting that Eomes regulates expression of both Il10 and Ifnγ directly. Consistent with this concept, the recruitment of RNA polymerase II to the Il10 promoter, an indicator of transcriptional activity, was reduced in Eomes-deficient CD4⁺ T cells (FIG. 3D).

### EOMES+T_{R}1 Cells are Dependent on Blimp-1, IL-27 and IL-10

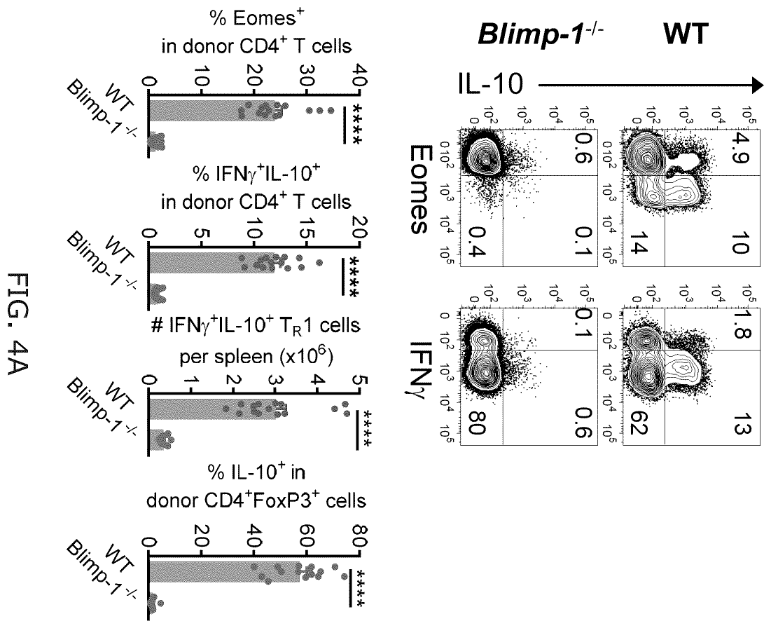

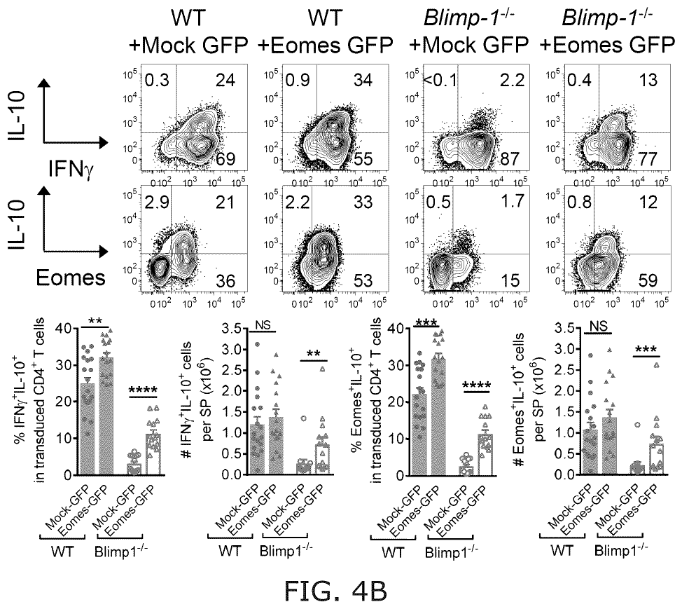

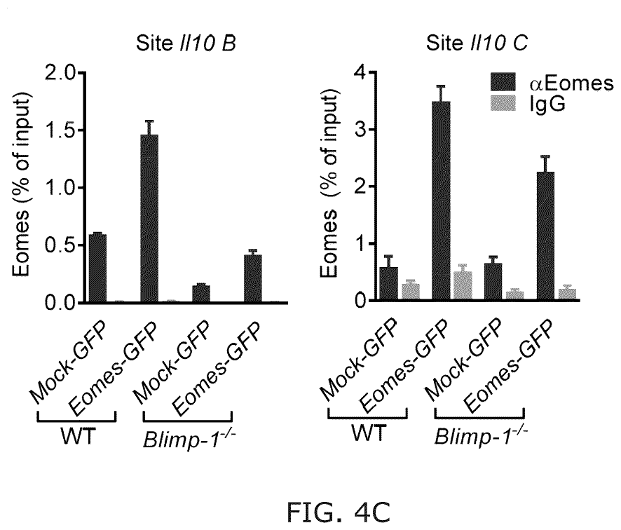

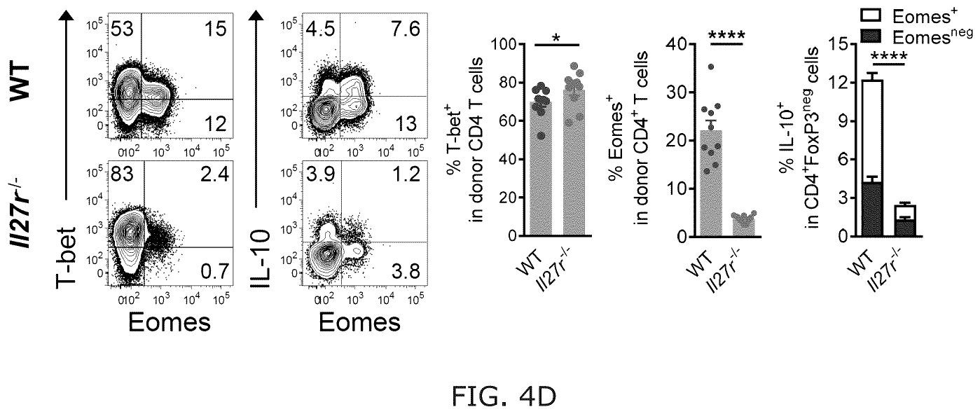

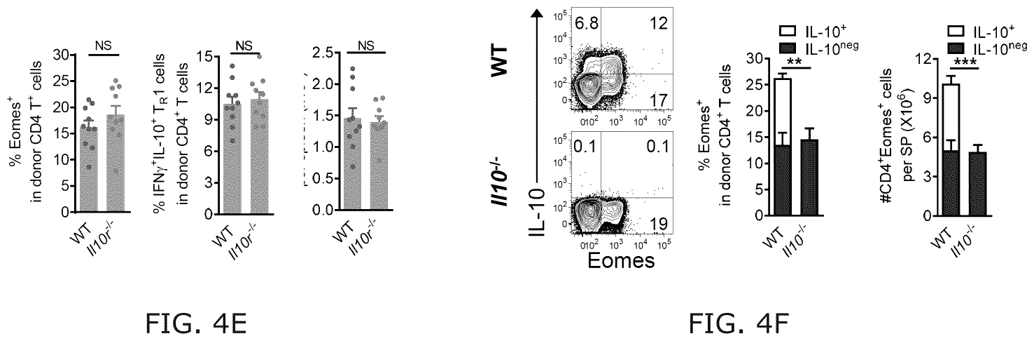

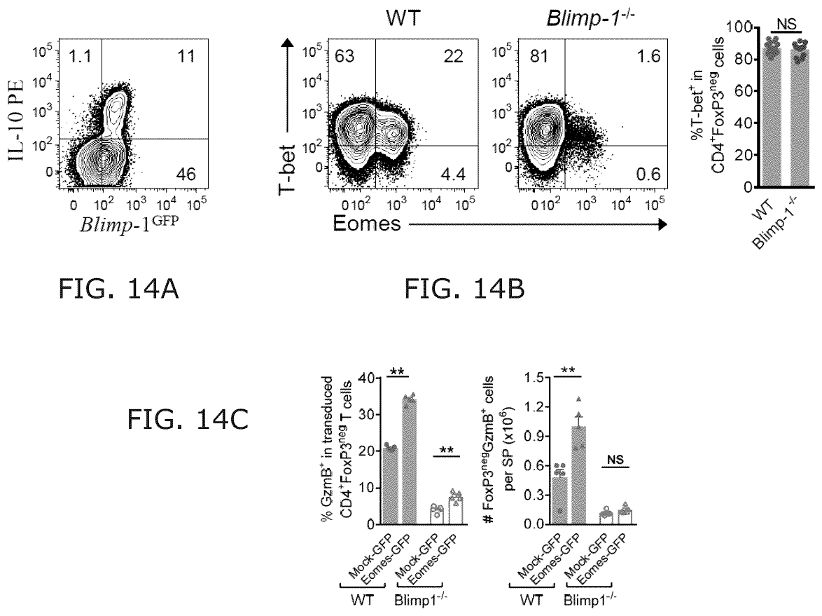

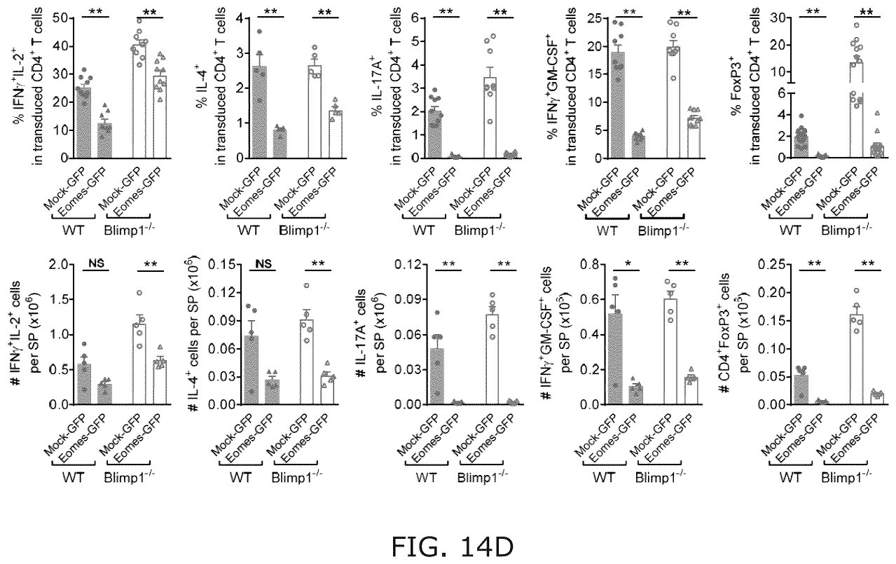

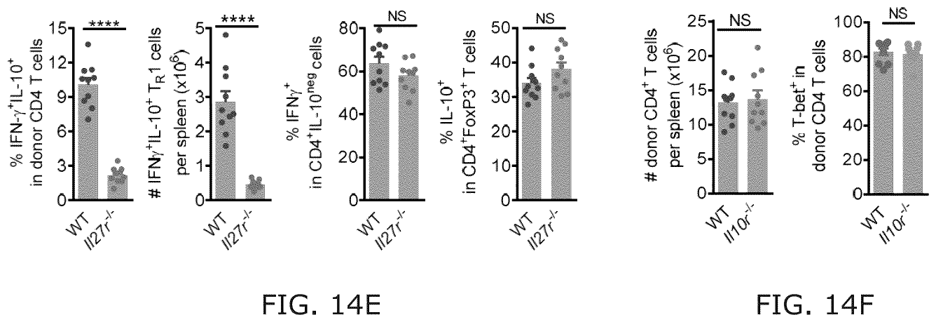

Blimp-1 is a well-defined transcriptional promoter of IL-10 in CD4⁺ conventional T and T_{reg} cells (6, 11, 21). Consistent with this notion, after BMT IL-10 production in all CD4⁺ T cells was confined to Blimp-1 expressing cells (FIG. 14A). Critically, conditional ablation of Blimp-1 (Prdm1^{fl/fl}×Lck-cre) in donor T cells resulted in a near complete loss of both IL-10 and Eomes expression in CD4⁺ T cells, demonstrating a near complete lack of T_{R}1 cells (FIG. 4A) while the expression of T-bet was not impaired (FIG. 14B). To elucidate the relative contribution of Eomes and Blimp-1 to the expression of IL-10, Eomes-transduced WT or Blimp-1^{-/-} CD4⁺ T cells were transferred into allogenic BMT recipients. Consistent with a critical role of Eomes in the differentiation of T_{R}1 cells, over-expression of Eomes in Blimp-1-deficient CD4⁺ T cells partially rescued their defective expression of IL-10 and GzmB and suppressed the expression of IL-2, IL-4, IL-17A, GM-CSF and FoxP3 after BMT (FIGS. 4B, 14C,D). Furthermore, Eomes transduction enhanced the recruitment of Eomes to the Il10 promoter regions both in WT and Blimp1^{-/-} CD4 T cells (FIG. 4C).

To test the role of IL-27 in the induction of Eomes⁺ T_{R}1 cells after BMT, the present inventors transplanted Il27r^{-/-} CD4⁺ T cells. Consistent with an important role for IL-27 in T_{R}1 induction, substantially decreased expression of Eomes was found in Il27r^{-/-} CD4⁺ T cells, and T_{R}1 cells were reduced by >80% (FIGS. 4D, 14E). In contrast, T-bet expression was increased in the absence of IL-27 signalling (FIG. 4D), and the development of CD4⁺IL-10^{neg}IFNγ⁺ conventional T_{H}1 cells or IL-10 production capabilities of T_{reg} cells were not impaired (FIG. 14E).

The present inventors next tested whether the differentiation of Eomes⁺ T_{R}1 cells was dependent on IL-10 itself. The expression of Eomes, T_{R}1 cells as well as T-bet was not reduced in Il10r-deficient CD4⁺ T cells (Il10r^{fl/fl}×Lck-cre) after BMT (FIGS. 4E, 14F), indicating that IL-10 signalling in T cells was not required for T_{R}1 cell differentiation. However, when I11-1-CD4⁺CD25^{neg} T cells were transplanted, Eomes+ cells were reduced (FIG. 4F), in line with the notion that IL-10 promotes T_{R}1 cell differentiation indirectly (22, 25). In summary, Eomes expression in T_{R}1 cells is downstream of IL-27 and Blimp-1 but does not depend on T cell intrinsic IL-10 signalling.

### EOMES+T_{R}1 Cells are Critical for Prevention of GVHD

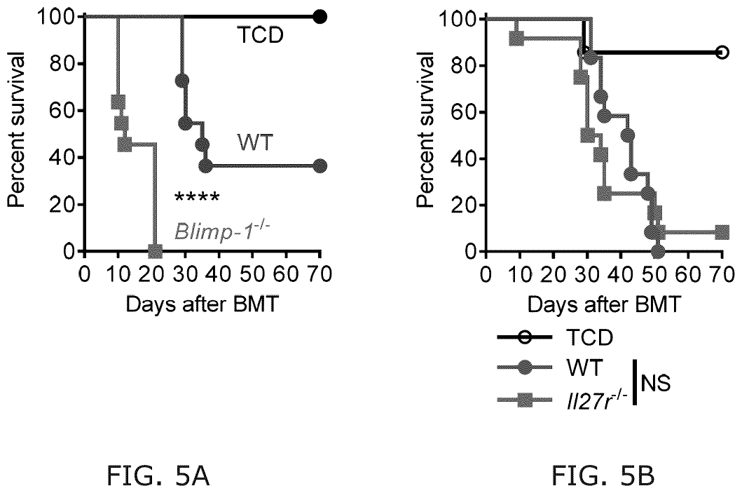

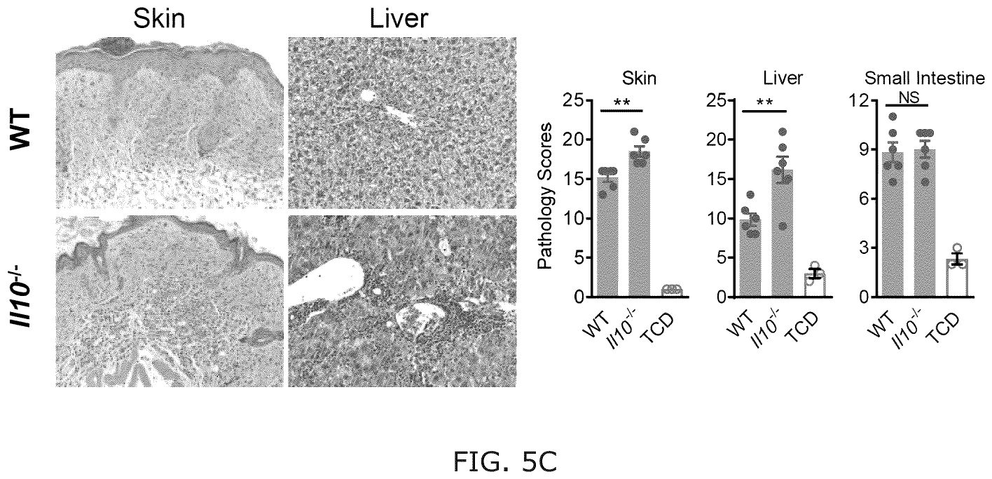

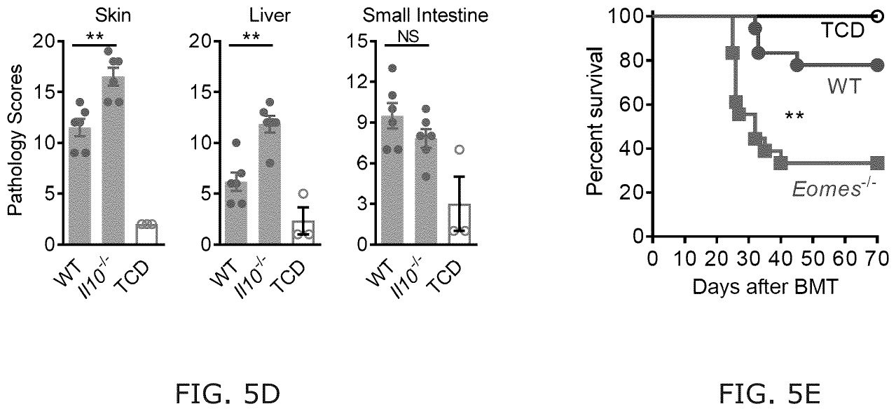

The present inventors next examined whether Blimp-i-deficient and Il27r-deficient CD4⁺ T cells would exacerbate GVHD due to impaired expression of Eomes and T_{R}1 cells. Whilst Blimp1 deletion exacerbated GVHD (FIG. 5A), IL-27R deletion did not (FIG. 5B). Of note, T_{reg} cells were increased and their IL-10 production was intact in recipients of Il27r^{-/-} CD4⁺ T cells (FIGS. 14E, 15A), consistent with compensatory regulatory pathways in the absence of T_{R}1. In contrast, Il10^{-/-} CD4⁺ T cells sustain comparable expression of Eomes in conventional T cells and T_{reg} cells (FIGS. 4F, 15B) after BMT and thus reflect a more relevant model to define the regulatory function of T_{R}1 cells in vivo. Consistent with the reduced frequency of T_{R}1 cells, enhanced GVHD was observed in the skin and liver in recipients of Il10^{-/-} CD4⁺CD25^{neg} T cells (FIG. 5C). These findings were confirmed by transplanting Il10^{fl/fl}×Lck-cre CD4⁺CD25^{neg} T cells, which also led to exacerbated GVHD in the absence of IL-10 producing T_{R}1 cells (FIG. 5D). Lastly, Eomes-1-CD4⁺CD25^{neg} T cells also resulted in increased GVHD, further confirming the important regulatory role of Eomes⁺ T_{R}1 cells after BMT (FIG. 5E).

### EOMES and T-Bet Cooperate to Generate T_{R}1 Cells

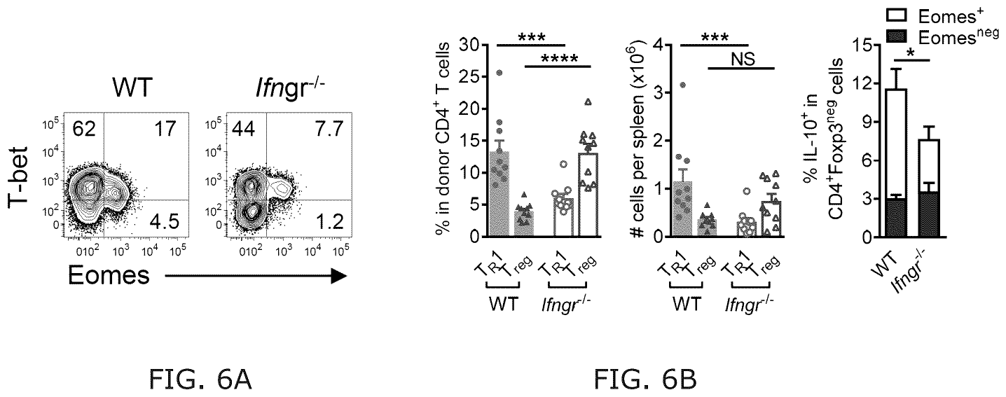

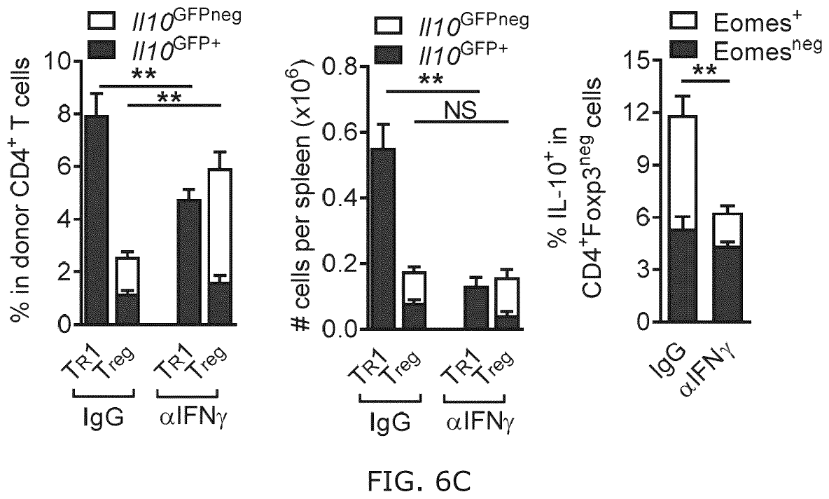

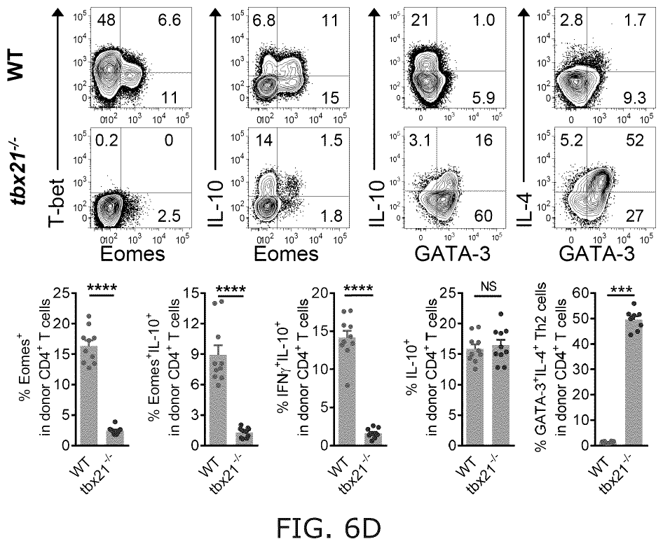

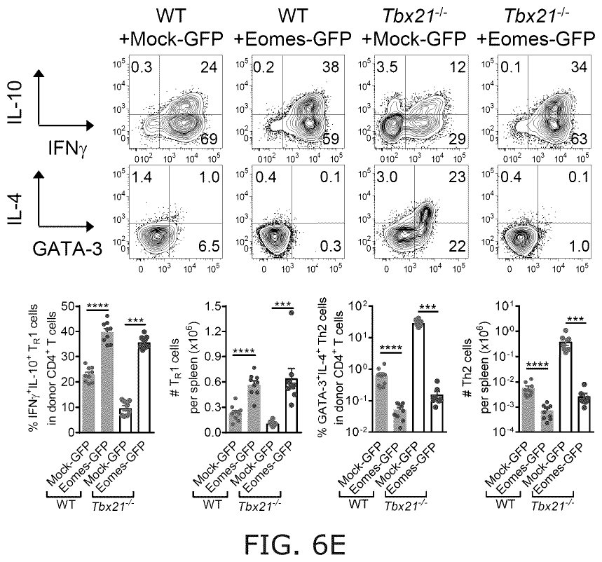

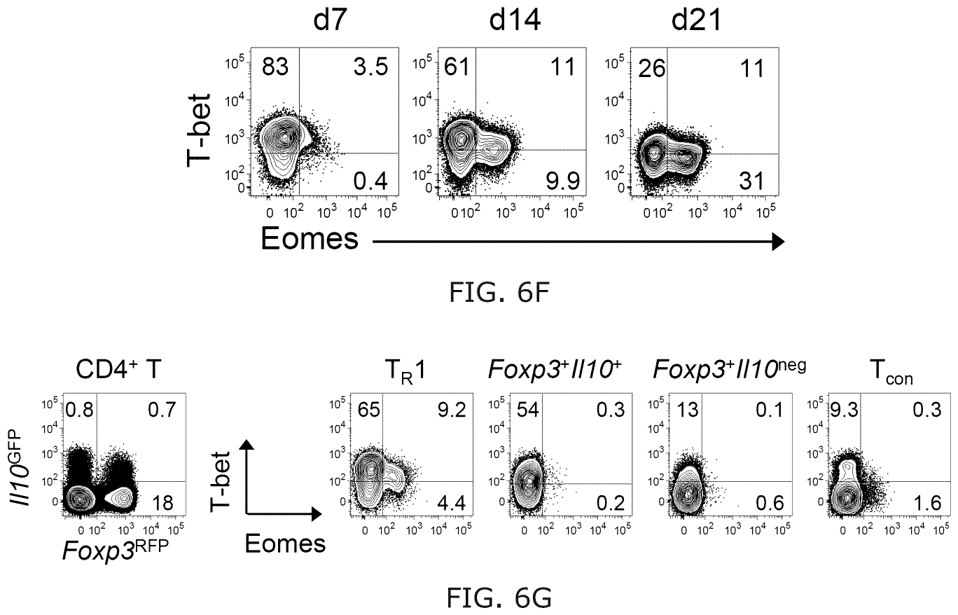

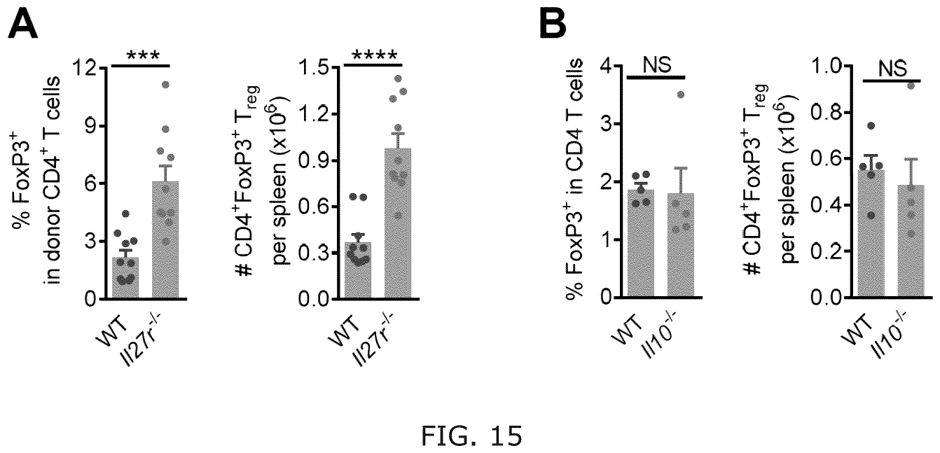

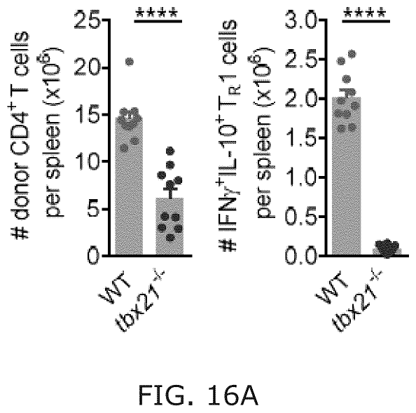

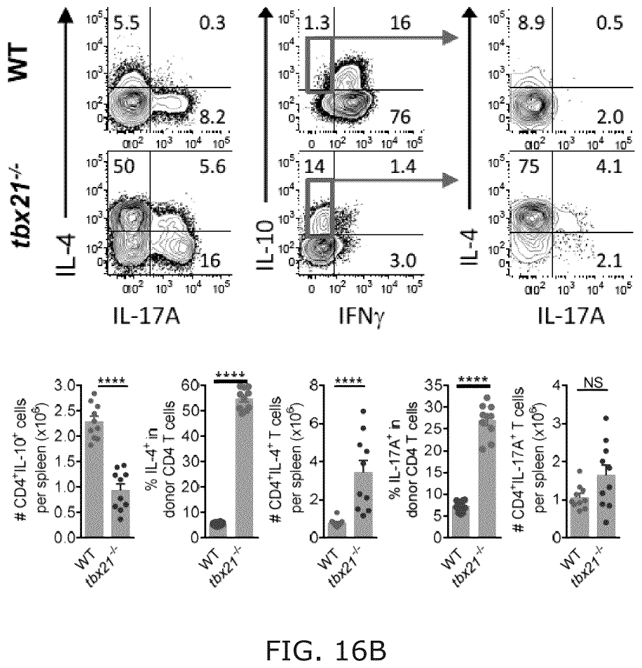

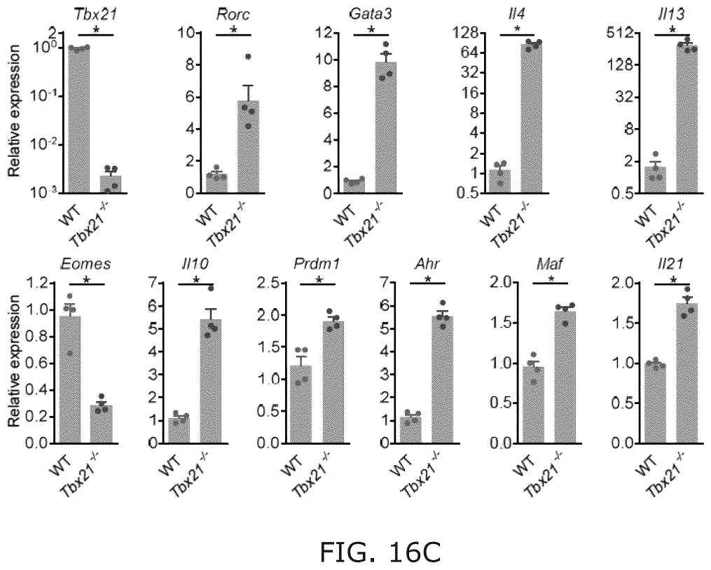

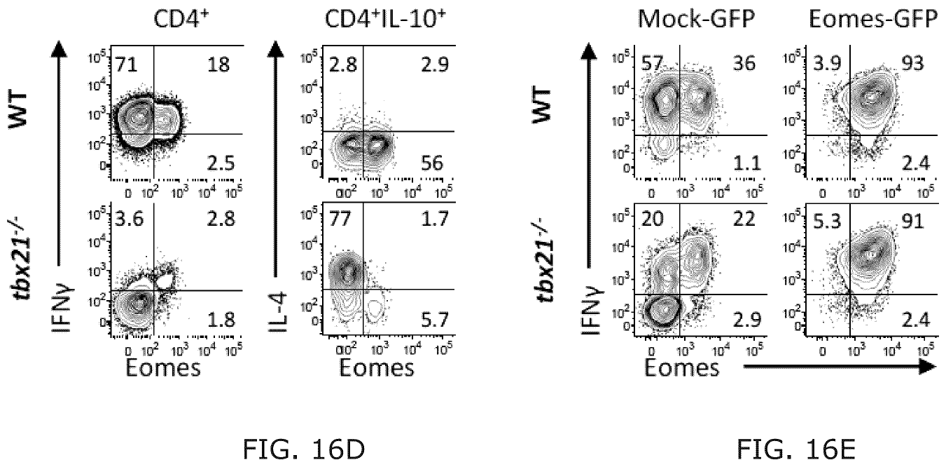

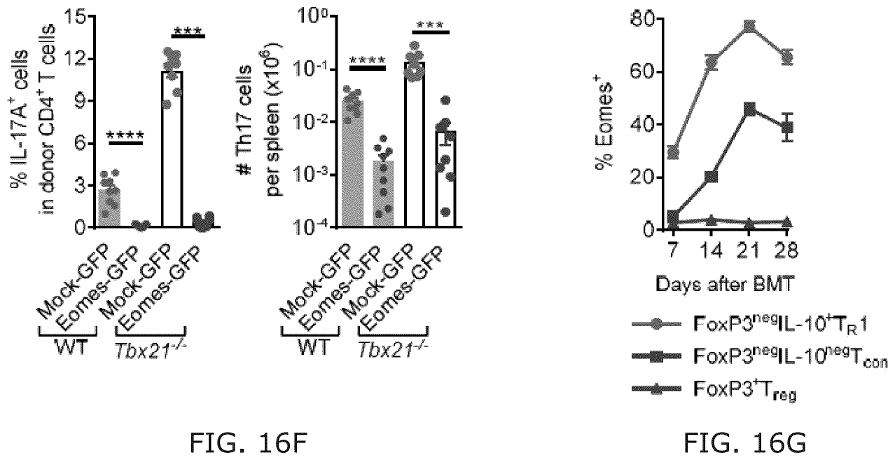

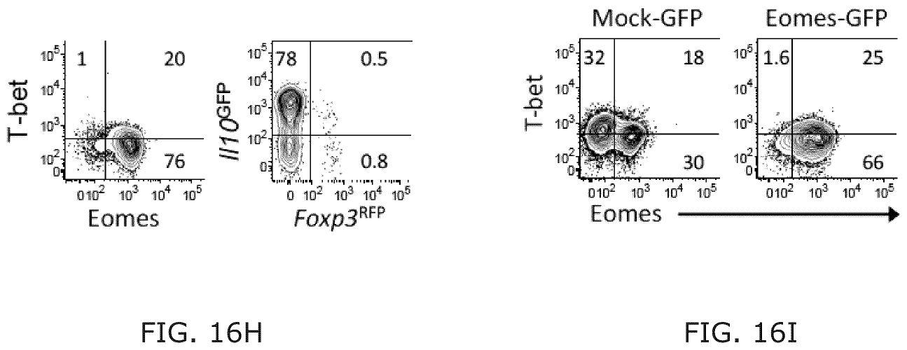

As co-expression of T-bet (encoded by Tbx2l) and Eomes had been observed in T_{R}1 cells after BMT, the present inventors wished to test the role of IFNγ signalling and T-bet in T_{R}1 cell development. Transplanting Ifngr^{-/-} donor T cells or neutralizing IFNγ resulted in reduced expression of T-bet and Eomes (FIG. 6A) with reduced expression of Eomes⁺ T_{R}1 cells and expanded T_{reg} cell populations (FIG. 6B, C). When Tbx21^{-/-} CD4⁺ T cells were transplanted during BMT, it was found that Eomes⁺ T_{R}1 cells were dramatically reduced (FIGS. 6D, 16A). Although overall frequencies of IL-10⁺CD4⁺ T cells were unaffected, the absolute numbers were reduced (FIG. 6D). Importantly, however, the majority of the Tbx21^{-/-}IL-10⁺CD4⁺ T cells did not express IFNγ but rather IL-4 and GATA3 or IL-17A, indicating that these cells had been diverted to T_{H}2 or T_{H}17 cells, respectively (FIGS. 6D, 16B). Gene expression analysis confirmed polarization of donor CD4⁺ T cells to T_{H}2 (Gata3, 114, I113) and T_{H}17 (Rorc, Ahr, I121) lineages in the absence of T-bet. The transcription of Il10 (from Th2 cells) was also increased (FIG. 16C). Notably, the residual Eomes⁺ population in Tbx21^{-/-} CD4⁺IL-10⁺ cells expressed IFNγ but did not express IL-4 (FIG. 16D). Thus, T-bet and IFNγ promote Eomes expression within the T_{R}1 lineage after BMT and, in concert with Eomes, repress alternate cell fates. To further understand the relative function of Eomes and T-bet in the differentiation of T_{R}1 cells, Tbx21^{-/-} CD4⁺ T cells were retrovirally transduced with Eomes. The over-expression of Eomes fully rescued the expression of IL-10, IFNγ, and IL-10⁺IFNγ⁺ T_{R}1 cells and correspondingly suppressed the expression of GATA-3⁺IL-4⁺T_{H}2 and IL-17A⁺ T_{H}17 cells (FIGS. 6E, 16E,F).

Next, the present inventors investigated whether there is a temporal and/or spatial collaboration between T-bet and Eomes during T_{R}1 cell development. First, Eomes expression in T_{R}1 cells was profoundly time-dependent after BMT (FIG. 16G), and CD4⁺ T cells transited from a T-bet^{hi} Eomes^{lo} to a T-bet^{lo}Eomes^{hi} state over time (FIG. 6F), correlating with the increasing frequency of T_{R}1 cells (FIG. 1E). Furthermore, after repeated exposure to high levels of alloantigen in vivo, the majority of donor CD4⁺ T cells had acquired Eomes (>95%) and converted to T_{R}1 cells (>70%) within four weeks of transfer into secondary BMT recipients (FIG. 16H). Consistently, over-expression of Eomes suppressed the expression of T-bet while promoting T_{R}1 cell differentiation (FIGS. 3B, 16I). T_{R}1 cells (Foxp3^{RFPneg}Il10^{GFP+}), found in low frequencies in naïve mice, also exhibited higher Eomes expression. This was specific to T_{R}1 cells as IL-10 producing T_{reg} cells (Foxp3^{RFP+}Il10^{GFP+}) expressed some T-bet but not Eomes (FIG. 6G). Collectively, these data show that both T-bet and Eomes are required for T_{R}1 cell differentiation, which is characterized by the initial up-regulation of T-bet, the acquisition of Eomes expression and the subsequent down-regulation of T-bet, resulting in a T-bet^{lo}Eomes^{hi} phenotype.

### Recipient Dc and Donor-Derived IL-27 Promote T_{R}1 Cell Development

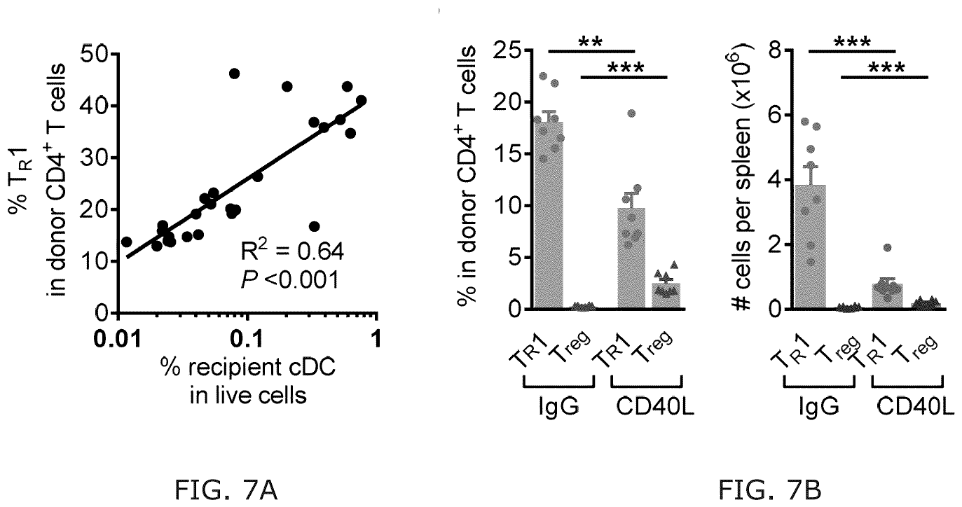

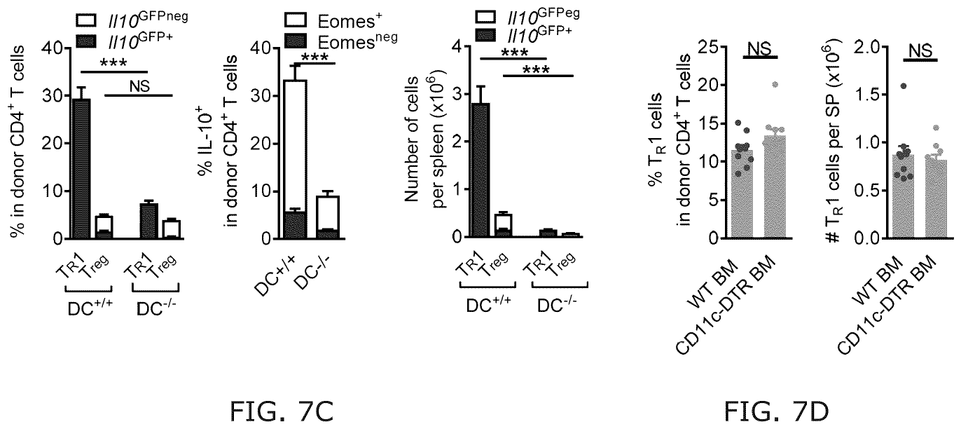

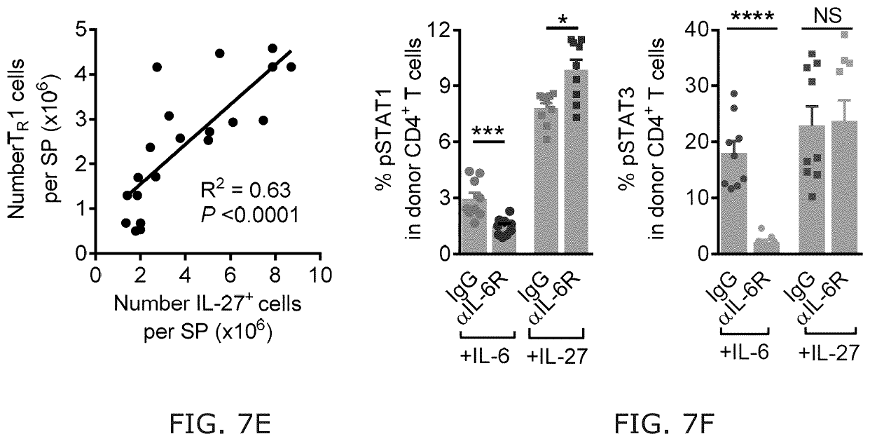

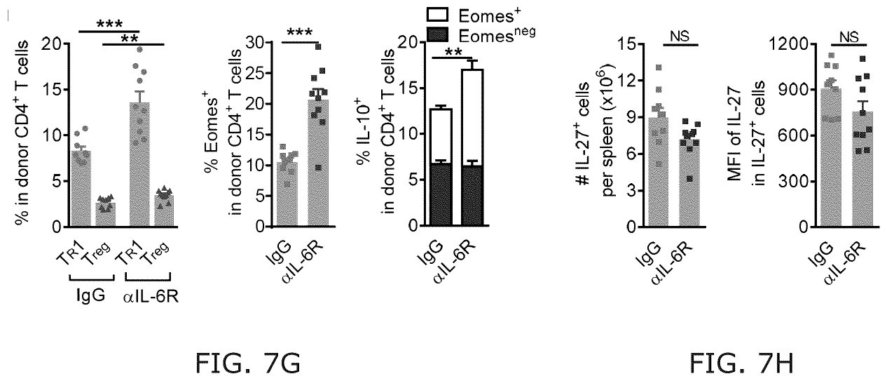

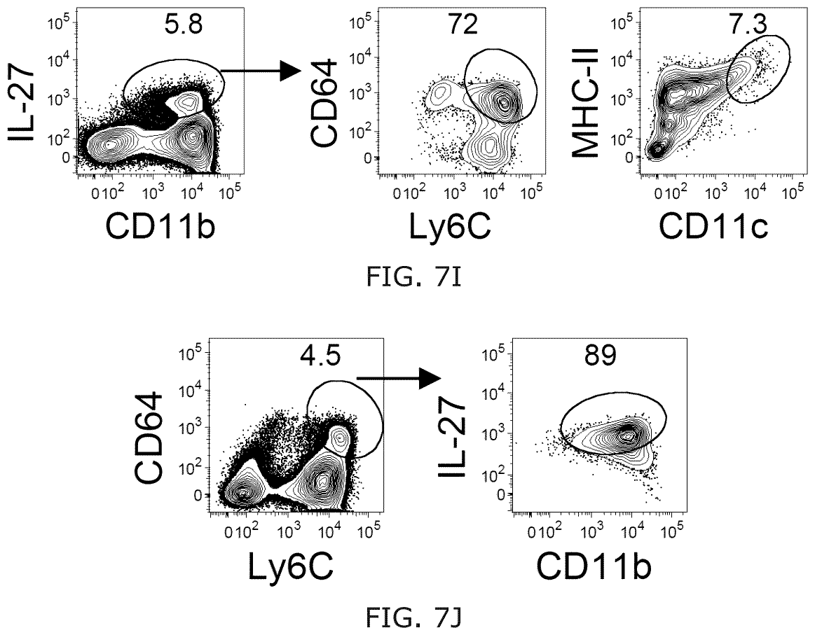

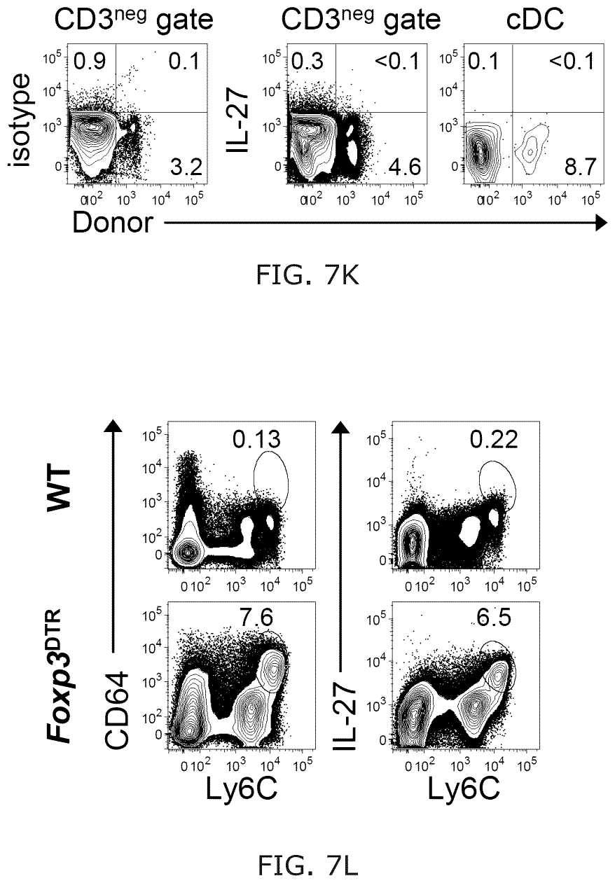

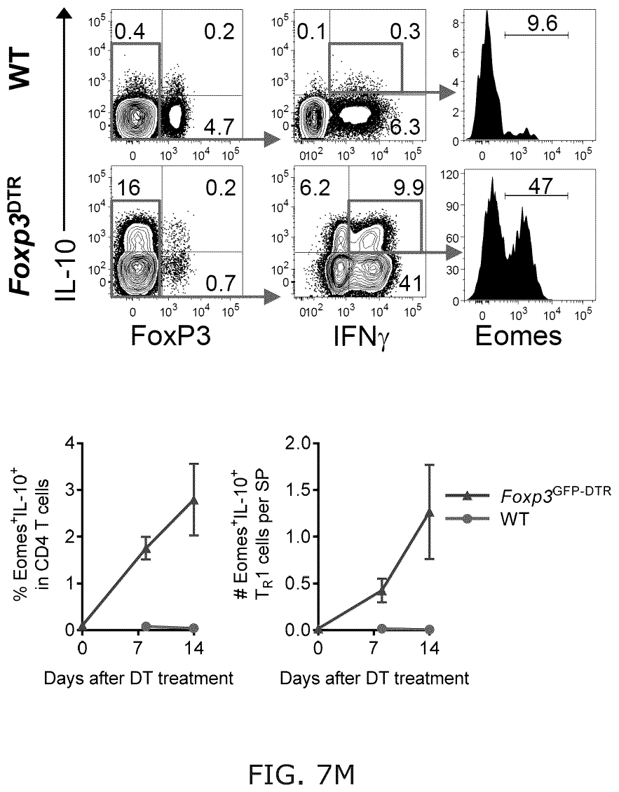

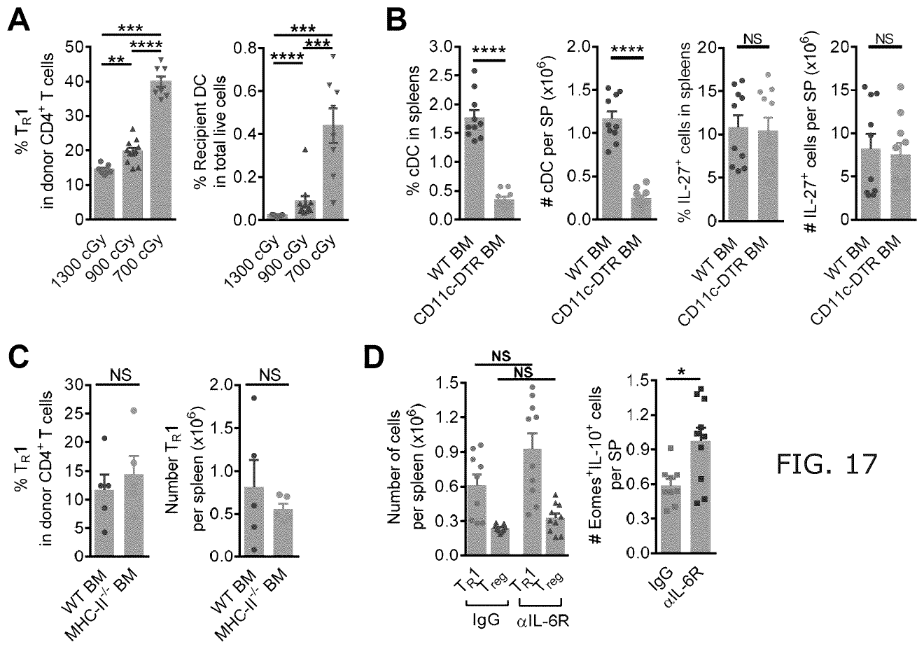

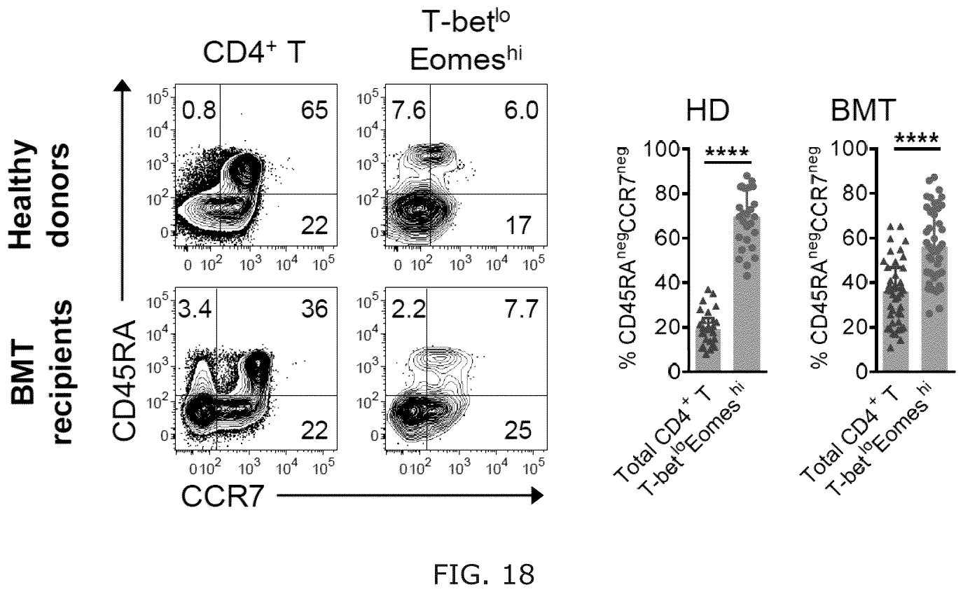

GVHD is initiated by recipient antigen presenting cells (APC) and is influenced by the intensity of conditioning, i.e. total body radiation (TBI) and chemotherapy dose-intensity, in part through inflammatory cytokine dysregulation (26, 27). The present inventors thus hypothesized that T_{R}1 cells may also be generated in an APC and conditioning-dependent fashion. The frequency of T_{R}1 cells in donor CD4⁺ T cells indeed correlated with the frequency of residual recipient conventional dendritic cells (DC) (FIG. 7A) and reduced intensity of TBI that favour the persistence of recipient DC (FIG. 17A). Blocking DC function by CD40L inhibition reduced T_{R}1 cells whilst favouring T_{reg} cell development (FIG. 7B). In line with this observation, depletion of both donor and recipient DC dramatically reduced the development of T_{R}1 cells early after BMT (FIG. 7C). While the proportions of T_{reg} cells were unaffected, absolute numbers were reduced, albeit much less dramatically than T_{R}1 cells (FIG. 7C). In contrast, the depletion of donor DC or inactivation of donor APC function in isolation did not impair T_{R}1 cell development (FIGS. 7D, 17B,C), indicating that recipient DC are required for the development of T_{R}1 cells early after BMT.

Consistent with the notion that Eomes⁺ T_{R}1 cells are dependent on IL-27 signalling and further confirming critical role of IL-27 in promoting T_{R}1 cell development, the present inventors found that the number of T_{R}1 cells significantly correlated with the number of IL-27⁺ cells in the spleen (FIG. 7E). As IL-27R and IL-6R share and compete for the same signalling component, gp130 (28), it was hypothesized that blocking IL-6R may favour IL-27R function. As expected IL-6R inhibition blocked STAT3 phosphorylation in response to IL-6 but not IL-27 (FIG. 7F). In contrast, IL-6R inhibition enhanced STAT1 phosphorylation in response to IL-27 early after BMT (FIG. 7F) and resulted in increased expression of T_{R}1 cells and a small increase in the frequencies of T_{reg} cells (FIGS. 7G, 17D). The enhanced STAT1 phosphorylation in response to IL-27 following IL-6R inhibition was not a result of an increase in the number of cells producing IL-27 itself or IL-27 production on a per cell basis (FIG. 7H). The present inventors next sought to identify the cellular sources of IL-27 after BMT. The majority of IL-27 (70-80%) was produced by Ly6C^{hi} donor macrophages (CD11b⁺, MHC II⁺, Ly6C^{hi}, F4/80^{hi}, CD64⁺ and CCR2⁺) with a more limited contribution from donor DC (CD11c⁺, MCH-II⁺) (FIG. 71). More than 80% of all Ly6C^{hi} donor macrophages were secreting IL-27 after BMT (FIG. 73). Depletion of donor DC did not impair the overall frequencies or numbers of IL-27⁺ cells (FIG. 17B), consistent with the lack of contribution by donor DC to T_{R}1 cell development. Lastly the present inventors demonstrated that recipient DC did not produce IL-27 early after BMT (FIG. 7K), suggesting that the requirement of recipient DC to T_{R}1 cell development relates to their capacity for alloantigen presentation and not IL-27 production. Thus donor macrophages appear the main producers of IL-27 and, in concert with the initial stimulation by recipient DC, drive Eomes-dependent T_{R}1 development after BMT.

To further understand the requirement of Eomes in T_{R}1 cell development, the expression of Eomes⁺ T_{R}1 cells was investigated in other models of immune pathology. To this end Foxp3^{GFP-DTR} mice were used to temporarily deplete T_{reg} cells, thereby causing autoimmunity (29-31). Indeed, depletion of T_{reg} cells from adult mice resulted in a dramatic increase in IL-27 producing Ly6C^{hi} macrophages (FIG. 7L) and critically, induced large numbers of Eomes⁺ T_{R}1 cells (FIG. 7M). Thus, the data presented herein demonstrate that different inflammatory conditions result in the development of Eomes⁺ T_{R}1 cells. Furthermore, the results presented herein demonstrate that defects in T_{reg} cells are associated with compensatory increases in Eomes⁺ T_{R}1 cells.

### Identification of T_{R}1 Cells in Humans

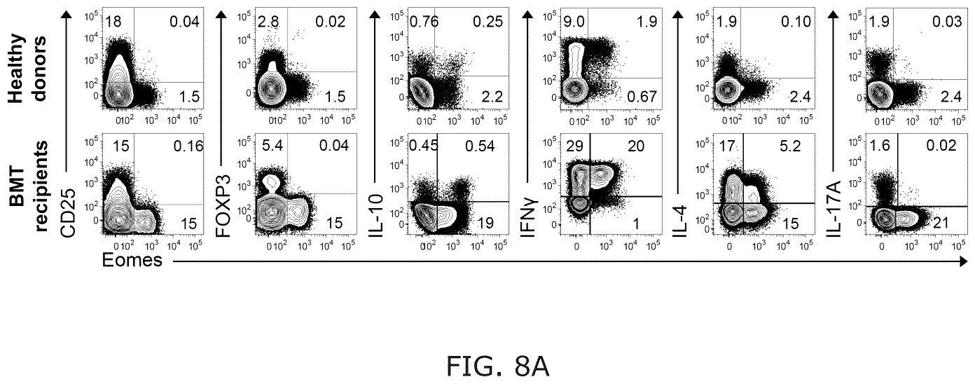

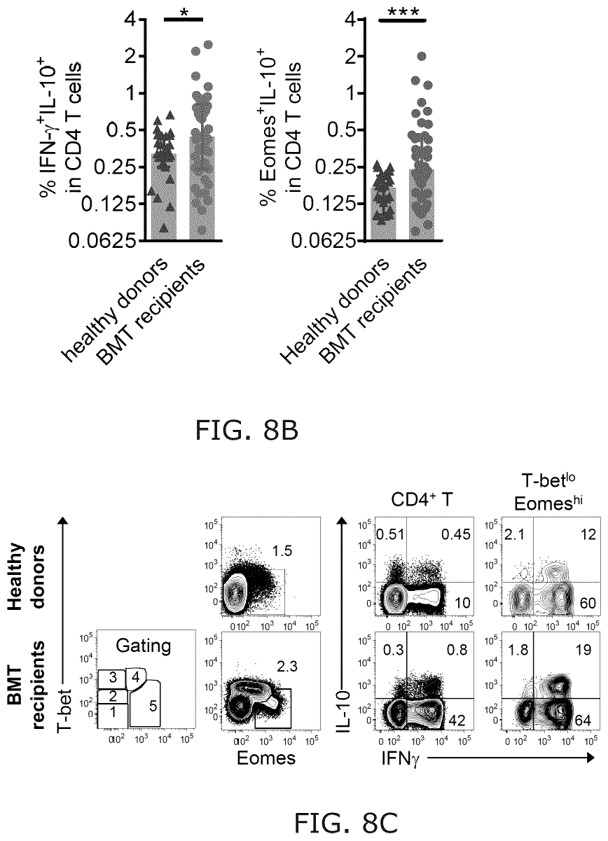

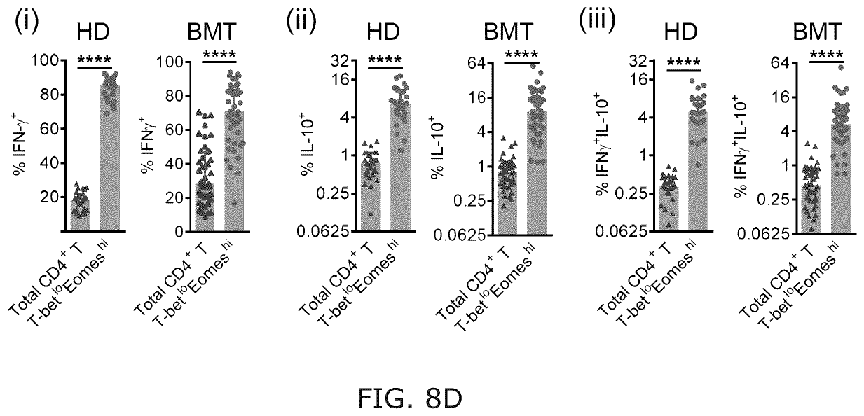

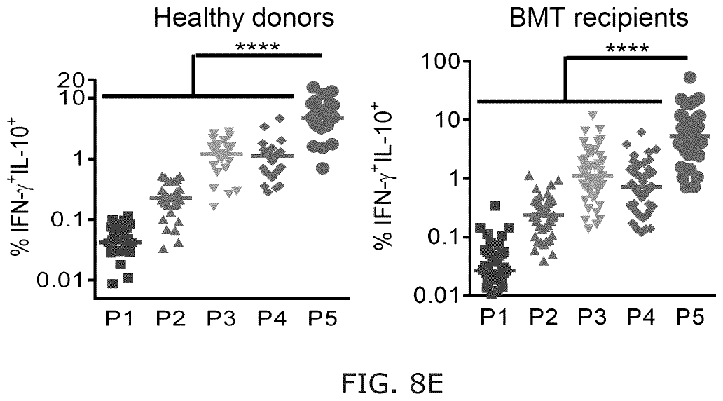

To validate whether the present findings from experimental BMT can be translated into humans, the expression of Eomes, IL-10 and other markers was analyzed in CD4⁺ T lymphocytes collected from healthy donors and BMT recipients. Eomes+CD4⁺ cells from healthy individuals as well as BMT recipients were CD25^{lo}, FOXP3neg, IFNγ^{hi}, IL-4^{lo} and IL-17A^{neg} and a proportion secreted IL-10 (FIG. 8A). Thus, human Eomes⁺IL-10⁺ cells show the characteristics of TR1 cells. Of note, compared to currently utilized IL-10⁺IFNγ⁺ staining methods, the use of Eomes in defining IL-10 positive T_{R}1 cells (Eomes⁺IL-10⁺) provides better discrimination of T_{R}1 cells between healthy donors and BMT recipients (FIG. 8B). Furthermore, the use of T-bet and Eomes expression defines populations with increasing proportions of IL-10⁺IFNγ⁺ T_{R}1 cells (FIG. 8C-E), consistent with the requirement for these transcription factors at different stages of differentiation both in steady state and after clinical BMT. IL-10⁺IFNγ⁺ T_{R}1 cells were enriched (>10 fold) in the T-bet^{lo}Eomes^{hi} population, which exhibited an effector memory (CD45RA^{neg}CC R7^{neg}) phenotype (FIGS. 8C-E, 18). Thus, consistent with the findings in the mouse model, after clinical BMT high Eomes and low T-bet expression in CD4⁺ T cells can be used to identify a population that is enriched for TR1 cells.

### SUMMARY

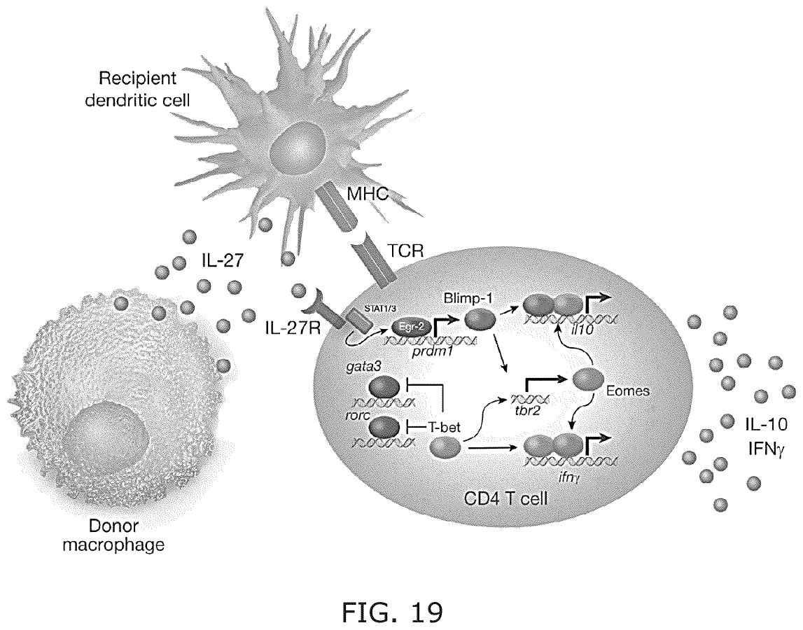

The present inventors demonstrate that Eomes acts together with Blimp-1 and specifically drives the development of T_{R}1 cells. Based on the data presented herein, a model for the differentiation of T_{R}1 cells after BMT is proposed as illustrated in FIG. 19. In this model, antigen presentation by recipient DC and macrophages-derived IL-27 provide the cellular and molecular cues for the development of T_{R}1 cells, inducing Blimp-1 expression, which initiates the transcription of Il10. Blimp-1 is also required for Eomes expression, and both factors act in concert, enabling stable IL-10 production and T_{R}1 cell differentiation. Concurrently, T-bet is required to suppress GATA3 and RORγt whilst driving IFNγ and Eomes expression ultimately leading to a T-bet^{lo} Eomes^{hi} phenotype, which can reliably identify T_{R}1 cells after BMT as well as in steady state in mouse and man.

There is still debate whether T_{R}1 cells constitute an independent lineage or simply represent IL-10 producing T_{H}1 cells. In particular, the lack of a master transcriptional factor for T_{R}1 cells has made progression of the field difficult (5, 13, 33). Multiple transcription factors, including Blimp-1, AhR and c-Maf are induced by IL-27 and have been shown to be critical for T_{R}1 cell differentiation (5-8, 10); however, none of them appear to be specific to the T_{R}1 lineage. Eomes is a T-box transcription factor which is more often than not coupled with T-bet in the biology of CD8⁺ T cells and NK cells (34, 35). Its roles in regulating functions of CD4⁺ T cells (36, 37) and suppressing T_{reg} and T_{H}17 cells differentiation have been described recently (38, 39). Here the present inventors demonstrate that IL-10⁺IFNγ⁺ T_{R}1 cells are uniquely dependent on Eomes. They found that Eomes bound to the Il10 and Ifnγ promoters. Similarly, it has been shown that Eomes also binds to the promoter of Gzmb (35), expression of which is another feature of T_{R}1 cells. Eomes over-expression was sufficient to promote IL-10 and GzmB and suppress other lineage-characteristic transcription factors (e.g. FoxP3, GATA-3, RORγt and BCL-6) and cytokines (e.g. IL-2, IL-4, IL-13, GM-CSF and IL-17A). Therefore, expression of Eomes and IL-10 within CD4⁺ T cells defines the T_{R}1 cell lineage.

Increasing data has suggested a close relationship between T_{R}1 and T_{H}17 cells linked via AhR, c-Maf and IL-21 (10, 23, 24, 40). However, T_{R}1 and T_{H}17 cells require different cytokines for their respective differentiation, IL-27/IL-10 for the former and IL-6/TGF-β/IL-23 for the later (12, 41-43). Multiple groups have independently shown IL-27 opposes the functions of IL-6/IL-23 in T_{H}17 differentiation (8, 28, 44). The data presented herein demonstrate that inhibition of IL-6R signaling favors IL-27 function and subsequent development of Eomes⁺ T_{R}1 cells. It is further shown that Eomes distinguishes T_{R}1 cells from other T_{H} lineages including T_{H}17 cells and its over-expression represses polarization to T_{H}17 cells. This is in line with the notion that Eomes suppresses T_{H}17 cell differentiation by directly inactivating Rorc and I117a promoters (39). A role for IL-27 in inhibiting T_{reg} reconstitution after BMT has also recently been reported (45), consistent with the counter-balanced T_{R}1 expansion seen here. There appears to be significant interplay between IL-6 and IL-27 (28), an effect also seen during GVHD. IL-6 inhibition has an intriguing capacity to enhance IL-27 responses and thereby to promote T_{R}1 cell differentiation, an effect likely contributing to clinical efficacy (46).

Eomes can be regulated by T-bet in a Runx-3 dependent manner and the differential expression of these two T-box transcription factors is critical for the differentiation of CD8⁺ T cells (47, 48). In line with this notion, the present inventors show that IFNγ signalling and T-bet expression were required for Eomes expression, demonstrating an important role of T-bet in the early phase of T_{R}1 cell development. Downstream of IL-27, Blimp-1 is critical for the expression of IL-10 in CD4⁺ T cells in various models (6-8, 21, 49). Here it is shown that Eomes⁺ T_{R}1 cells are regulated by both Blimp-1 and T-bet, consistent with a recent report that demonstrated close collaboration between Blimp-1 and T-bet in CTL generation (50). In addition, binding of Blimp-1 to the Eomes promoter in CD8⁺ T cells during viral infection has been described (32), suggesting that Blimp-1 not only regulates IL-10 expression directly but also contributes to the induction or maintenance of Eomes expression in T_{R}1 cells. Notably, both Blimp-1 (6) and Eomes bind to the Il10 locus, and the activity of both is required to promote efficient T_{R}1 differentiation and Il10 expression. Interestingly, similar to Eomes, Blimp-1 is not only required for IL-10 expression but also for granzyme B (51). The present inventors also confirmed that IL-10 itself contributes to T_{R}1 cell differentiation, a T cell extrinsic effect likely via myeloid cells (22, 25). Overall these data suggest that the functional interactions between Blimp-1, T-bet and Eomes are important for the differentiation of CD4⁺ T cells and T_{R}1 lineage in particular.

The present inventors consider that the identification of the bona fide transcriptional and cellular control of T_{R}1 cell development will permit therapeutic utilization of T_{R}1 cells in transplantation and other diseases where excessive and aberrant immunity results in immune pathology.

### MATERIALS AND METHODS

Study design. Female C57BL/6 (B6.WT, H-2b, CD45.2), B6.SJL-Ptprca (PTPrca, H-2b, CD45.1) and B6D2F1 (H-2b/d, CD45.2) mice were purchased from the Animal Resource Center (Perth, WA, Australia). B6 Il27r^{-/-} and Tbx21^{-/-} mice were obtained from the Jackson Laboratory (Bar Harbor, ME, USA). B6 Blimp-1^{GFP}(52), Il10^{GFP}×Foxp3^{RFP}(19, 20), Foxp3^{GFP}, Foxp3^{GFP-DTR}, Il10^{-/-}, Ifnγr^{-/-}, MHC-II^{-/-}, Il10r^{fl/fl}×Lck-cre(53), Il10^{fl/fl}×Lck-cre(54), Prdm1^{fl/fl}×Lck-cre (Blimp-1^{-/-})(51), CD11cDOG and DBA2×B6.CD11cDOG mice were bred at the QIMR Berghofer Medical Research Institute animal facility. B6 Eomes^{fl/fl} mice were derived from the Eomes^{floxed/mcherry} mice previously generated by GTB and described in (55). The Eomes^{floxed/mcherry} mice were crossed to the B6.129S4-Gt(ROSA)26Sor^{tm2(FLP*)Sor}/J line which induces FLP-mediated recombination to remove the mCherry/Amp cassette to generate the Eomes^{floxed} line. Removal of the Frt sites (and hence the IRES-Cherry cassette) was detected using primers (a) 5′-ggacttggggagccaaaa-3′ (forward) and (b) 5′-cacatctgtaaccgcagcat-3′ (reverse) (deleted allele, 306 bp). The primers (c) 5′-agtcggtttgagctggtgac-3′ (forward), (d) 5′-tttggaacagcctccaaatc-3′ (reverse) were used to detect the wild-type (339 bp) and floxed allele (421 bp) while primer (e) 5′-AAGGGGAAGGGTGGTTAGAA-3′ (reverse) was used to detect the floxed allele (1941 bp) and germline deletion (587 bp). This Eomes^{floxed} line was subsequently crossed with Cd4-cre or Lck-cre mice to generate T cell restricted Eomes^{-/-} offspring. All recipient mice were used between 6 and 10 week of age and age matched female donor mice were used. Mice were housed in microisolator cages and received acidified autoclaved water (pH 2.5) after BMT. All animal studies were performed in accordance with the QIMR Berghofer Medical Research Institute Animal Ethics Committee. The inventors chose sample sizes based on estimates from initial and previously published results in order to ensure appropriate power. As stated in Figure legends and wherever possible, n values were derived from individual mice from replicated experiments.

Bone marrow transplantation. BM (B6.CD45.1⁺ or where indicated) was T cell depleted and splenocytes processed to CD3⁺ or CD4⁺ T cells as described previously (55). On day −1, recipient mice received 1100 cGy (B6D2F1), 1000 cGy (B6), 900 cGy (CD11c-DOG×DBA/2 F1) or otherwise specified doses of total body irradiation ([137Cs] source at 108 cGy/min), split into two doses separated by 3 h. On day 0, recipients were transplanted with 5−10×10⁶ BM cells with or without 1−2×10⁶ T cells (CD3⁺ or CD4⁺). Intraperitoneal injections of rat-anti-mouse IFNγ (XMG1.2, produced in house, 1 mg/dose, 3 times per week), hamster-anti-mouse CD40L (MR1, BioXcell, 500 ug/dose, d0, +2, +4, +6), rat-anti-mouse IL-6R (MR16-1, Chugai Pharmaceutical Co, Japan, 500 ug/dose, d-1, +3, +7) and control mAb were administered to recipients. In some experiments, CD11c-DOG mice (in which diphtheria toxin (DT) receptor is driven off the CD11c promoter) were used as BM donors. Recipients were given intraperitoneal injections of DT (160 ng/dose, 3 times per week) after BMT to deplete donor DC. For depletion of recipient DC, B6.CD11c-DOG×DBA/2 F1 mice were used as recipients and treated with DT on d-3, −1, 0, +1, +3, +5, +7.

T_{reg} depletion. For depletion of T_{reg}, age-matched recipients (B6.WT or B6.Foxp3^{GFP-DTR}) were given intraperitoneal injections of DT (160 ng/dose, 3 times per week) for up to 2 weeks.

Histology. GVHD target tissues (skin, liver and small intestine) were taken, preserved in 10% formalin, embedded in paraffin, processed to 5-mm-thick sections and H&E-stained. The sections were examined in a blinded fashion using a semi-quantitative scoring system and images acquired as previously described (56, 57).

Flow Cytometry. Single cell suspensions were processed and stained, cells were analyzed on a LSR Fortessa cytometer (Beckman Dickinson) and data were processed using FlowJo Version 9.0 (TreeStar). Cell sorting was performed using a FACSAria or Moflo.

Clinical analysis. Peripheral blood was collected from healthy donors (n=27) or patients (d60 after BMT) of an observational study (n=18) and a phase III clinical trial (ACTRN12614000266662) (n=25). All studies were approved by the institutional ethics committee and all subjects signed informed consent. Peripheral blood mononuclear cells (PBMC) were purified from whole blood using Ficoll-paque centrifugation and stained immediately.

Gene expression analysis. Total RNA was extracted with the RNeasy Micro kit (Qiagen, Netherlands) and gene expression determined using TaqMan GE assays (Applied Biosystems, MA, USA). All measurements were run in parallel with the housekeeping gene Hprt. All primers/probe mixtures were purchased from Applied Biosystems.

Statistics. Results from mouse experiments are presented as mean±SEM and the Mann-Whitney U test used for comparisons. Results from clinical samples are presented as median f interquartile range and Mann-Whitney U test used for comparisons. Survival is estimated and plotted using Kaplan-Meier methods, and the difference between subgroups is estimated using log-rank methods. Ordinary least squares method is used in the linear or semi-log regression analysis. A two-sided p value 0.05 is considered statistically significant. Statistical analyses are performed using Prism Version 6 software (GraphPad). NS=not significant, *P<0.05; **P<0.01;***P<0.001; ****P<0.0001.

Lymphocyte isolation from small intestine and liver. Small intestine were cut into 3-5 mm pieces, washed with PBS, incubated in Ca/Mg-free PBS containing EDTA (Chem-Supply, 5 mM) for 30 min at 37° C. with constant agitation. Cells were isolated by passing through 100 μm cell strainer and kept as fraction 1. The remaining sample was further treated with RPMI containing DNase (5 μg/mL) and Collagenase 4 (Sigma-Aldrich, 5 μg/mL) for 30 min at 37° C. and cells were isolated as fraction 2. Fraction 1 and 2 were combined and mononuclear cells isolated by Percoll density gradient centrifugation. After removal of gall bladder, liver was perfused with PBS, processed to single cell suspensions and mononuclear cells isolated by Percoll density gradient centrifugation (65).

In vitro suppression assays. T_{R}1, T_{reg} and T_{con} cells were FACS sorted on the basis of Il10^{GFP} and Foxp3^{RFP} from spleens d14 after BMT and in vitro suppression assays performed as previously described (64), with natural T_{reg} cells from naïve animals used as positive control. Percent of suppression is calculated as following: ((percent divided cells of T_{con} alone−percent divided cells of T_{con} with suppressors)/percent divided cells of T_{con} alone)*100.

Analysis of GVHD suppression. B6D2F1 recipients were transplanted with FACS sorted CD4⁺CD25^{neg} T cells (B6.WT or Il10^{-/-}) and BM (Il10^{-/-}) and monitored for systemic GVHD as described before (64). When acute GVHD was established (d7 after BMT), T_{R}1 cells FACS sorted from spleens and liver of a concurrently performed BMT (d14) were infused to one group of recipients (1×10⁶ per mouse), which were devoid of T_{R}1 cells (FIG. 1H).

Characterisation of sorted T_{R}1 cells. B6D2F1 were transplanted with CD3⁺T cells (Il10^{GFP}Foxp3^{RFP}) and spleens processed and sorted for T_{R}1, T_{reg} and T_{con} cells 14 days after BMT. Cells were stained for transcription factors immediately after sort and cytokines analysed by ICS. Sorted cells (10⁶/mL) were stimulated in 96 well plates with PMA (50 ng/mL) and ionomycin (500 ng/mL) for 24 hours and cytokines in culture supernatant quantified using the BD Cytometric Bead Array system (BD Biosciences).

Intracellular cytokine staining. Before intracellular cytokine staining (ICS) of T cells, single cell suspensions were stimulated for 4 hours with phorbol myristate acetate (PMA) (Sigma-Aldrich, 50 ng/mL) and ionomycin (Sigma-Aldrich, 500 ng/mL for murine cells, 1 μg/mL for PBMC) in the presence of brefeldin A (Biolegend). Before IL-27 staining, the cells were stimulated for 4 hours with lipopolysaccharide (Integrated Sciences, 1 ug/mL) in the presence of brefeldin A. The Live/Dead Fixable Dead Cell staining kit (Molecular Probes) was used to exclude dead cells. The cells were processed using the Foxp3/Transcription Factor Fixation/Permeabilization kit (eBiosciences) as per the manufacturer's instructions.

STAT Phosphorylation. Spleens were processed to single cell suspensions, surface stained, followed by stimulation with rmIL-6 (100 ng/mL, eBioscience) or rmIL-27 (100 ng/mL, Biolegend) at 37 degrees for 5 or 15 minutes respectively, then processed with BD Phosflow kit (BD Biosciences) before FACS analysis.

mAbs used in mouse experiments. The following mAbs were purchased from Biolegend: CD44 Brilliant Violet (BV) 421 (IM7), CD62L Alexa Fluor (AF) 700 (MEL-14), CD49b AF647 (HMa2), Integrin α4β7 PE (DATK32), CD69 Pacific Blue (H1.2F3), CD279 (PD-1) PECY7 (RMPI-30), PD-L1 PE (10F.9G2), CXCR3 PE (CXCR3-173), GITR APC (YGITR765), DNAM-1 AF647 (TX42.1), CD223 (LAG3) PE (C9B7W), ICOS PE (7E.17G9), CD103 Pacific Blue (2E7), CD54 PE (3E2), CD11b PerCP/Cy5.5 (M1/70), Ly6C Pacific Blue (HK1.4), Ly6G APCCY7 (1A8), IA/IE FITC (M5/114.15.2), CD11c PE (N418), CD64 PECY7 (X54-5/7.1), F4/80 AF700 (BM8), IL-27 AF647 (MM27-7B1), IL-4 PE (11B11), IL-10 PE or APC (JES5-16E3), IL-2 PE (JES6-5H4), GM-CSF PE (MP1-22E9), IFNγ Pacific Blue (XMG1.2), IL-17A AF700 (TC11-8H10.1), FOXP3 AF647 (150D) or AF700 (FJK-16s), T-bet AF647 or PE (4B10), Helios Pacific Blue (22F6). The following mAbs were purchased from BD Bioscience: CD25 PE (7D4), CD28 biotin (37.51), BCL-6 AF647 (K112-91), pSTAT3 (pY705) PE (4/P-STAT3) and pSTAT1(pY701) PerCP/Cy5.5 (4a). The following mAbs were purchased from eBioscience: GARP PE (YGiC86), TIGIT efluor 660 (GIGD7), CD122 PerCP-eFluor 710 (Tm-β1), AhR (Aryl hydrocarbon receptor) PE (4MEJJ), GATA3 PE (TWAJ), RORγt PE (B2D) and Eomes PECY7 (Dani1mag). The following mAbs were purchased from R&D Systems: Neuropillin PE (761705), CCR2 APC (475301), anti-human TGF-β1 biotin (polyclonal) and Ki-67 PECY7 (B56). Anti-human Granzyme B (GB12) APC was purchased from Invitrogen.

mAbs used for PBMC staining. The flowing mAbs were purchased from Biolegend: TCRaβ PerCP/CY5.5 (IP26), CD4 AF700 (RPA-T4), CD8 APCCY7 (SK1), CD25 PECY7 (BC96), CD197 BV421 (G043H7), CD45RA FITC (HI100), IL-4 BV421 (MP4-25D2), IFN-γ PECY7 (4S.B3), IL-10 PE (JES3-19F1), IL-17A BV605 (BL168), T-bet AF647 (4B10), FOXP3 PE or AF647 (150D) The flowing mAbs were purchased from BD Bioscience: CD3-Brilliant Blue 515 (UCHT2), CD3 V500 (UCHT2), CD45 V500 (HI30) and CD127 BV605 (HIL-7R-M21). The following mAbs were purchased from eBioscience: CD279 (PD-1) PE (12-2799-42) and Eomes PE-eFluor 610 (WD1928).

ChIP and real-time PCR analysis. 5−7×10⁶ T_{R}1 cells (Il10^{GFP+}Foxp3^{RFPneg}) or CD4⁺ T cells were fixed, lysed and then sonicated to yield 0.5 kb DNA fragments, and 1% of the chromatin preparation was set aside as the input fraction. The chromatin was then immunoprecipitated with anti-Eomes (ab23345, Abcam), anti-pol II (ab817, Abcam) and rabbit IgG (ab37415, Abcam). DNA were isolated using the PCR purification/Gel Extraction kit (QIAGEN) and the SYBR Green PCR Kit was used for real-time PCR detection of the immunoprecipitated targets. Primers (A-C) were designed to detect DNA sequences containing T-box conserved sites (TCACACCT) flanking 0-2 kb upstream of the transcription start site (TSS) of the W/10 promoter (FIG. 3D). The primer sets are listed in table S1. The data are presented as percent of immunoprecipitated target sequences relative to input chromatin.

T_{H} cell polarization. MACS purified CD4⁺ T cells (>90% pure) were resuspended in IMDM (10⁶ cells/mL) with 10% FBS, 2 mM L-glutamine, 50 mM 2-ME, 100 U/mL penicillin, and 100 mg/mL streptomycin sulfate, and stimulated with plate-bound CD3 (3 ug/mL) and CD28 (1 ug/mL) for 4 days. The following combinations of cytokines were added for the polarization of T_{H}1: rhIL-2 (100 U/mL), IL-12p70 (10 ng/mL) and anti-IL-4 (clone 11B11, 10 μg/mL); T_{H}2: rhIL-2 (100 U/mL), rmIL-4 (eBioscience, 40 ng/mL) and anti-IFNγ (clone XMG1.2, 10 ug/mL); T_{H}17: IL-6 (30 ng/mL), TGFβ1 (5 ng/mL), IL-23 (15 ng/mL), IL-1β (20 ng/mL), TNFα (20 ng/mL), anti-IL-2 (clone JES6-1A12, 10 μg/mL), anti-IL-4 (10 μg/mL) and anti-IFNγ (10 μg/mL); T_{R}1 cells: rmIL-27 (Biolegend, 50 ng/mL) and TGFβ (1 ng/mL); induced T_{reg}: rhIL-2 (100 U/mL), TGFβ1 (10 ng/mL), Rapamycin (100 ng/mL). Cells were collected for analysis between day 4 and 7.

Retrovirus production and retroviral transduction. Envelope expressing plasmid (EcoPak) and vectors (MSCV-IRES-GFP or MSCV-Eomes-IRES-GFP) were used to transiently transfect HEK293T cells in the presence of GeneJuice (Novagen), and viral supernatants stored in −80° C. Retrovirus was centrifuged onto RetroNectin (Takara, 32 μg/mL)-coated plates for 1 h at 3000 g at 4° C. CD4⁺ T cells were stimulated with plate-bound CD3 (2C11, produced in house, 10 μg/mL) and CD28 (N3751, produced in house, 1 μg/mL) for 20-24 h before cultivating with the retrovirus in the presence of rhIL-2 (Aldesleukin, 100 U/mL) and Polybrene (Sigma-Aldrich, 16 μg/mL) for 4 h. Cells were then washed and cultured in the presence of rhIL-2 (100 U/mL) for 3-4 days, FACS sorted to GFP^{hi} population and allowed for further expansion 1-2 days for subsequent experiments.

In vitro generation of Eomes transduced T_{R}1 cells. Retrovirally transduced CD4⁺ T cells were resuspended in IMDM (10⁶ cells/mL) with 10% FBS, 2 mM L-glutamine, 50 mM 2-ME, 100 U/mL penicillin, and 100 mg/mL streptomycin sulfate, and stimulated with plate-bound CD3 (1 μg/mL) and CD28 (1 ug/mL) in the presence of rhIL-2 (50 U/mL) and rmIL-27 (50-100 ng/mL) for 20-24 h and then rested in culture with IL-2 and IL-27 as above for 4-5 days.

### Experimental 2

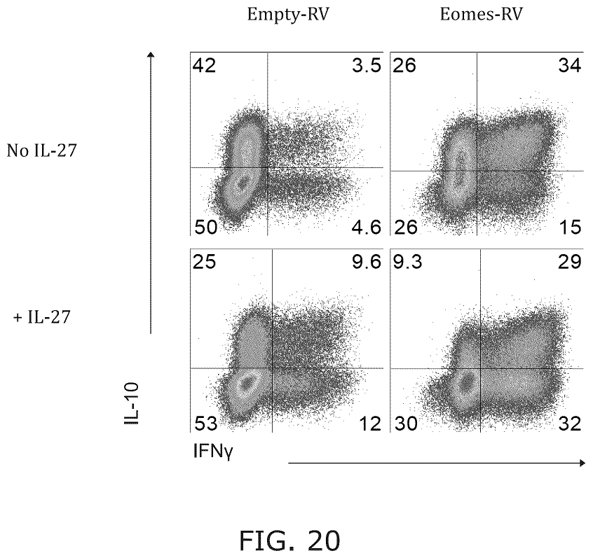

IL-27 is a critical cytokine for the generation of T_{R}1 cells both in vivo and in vitro (59, 60) which functions through the induction of Eomes expression (59). The present inventors hypothesized that Eomes over-expressing CD4⁺ T cells can be polarized to T_{R}1 cells in the absence of IL-27. Indeed, the expression of IL-10⁺IFNγ⁺ T_{R}1 cells in Eomes-RV transduced CD4⁺ T cells becomes independent of IL-27 (FIG. 20). In contrast, IL-27 is still required for the induction of T_{R}1 cells in Empty-RV transduced cells (FIG. 20). In addition to the simplification of generating the T_{R}1 cells product, the removal of IL-27 from cell culture also avoids unwanted effects of this multifaceted cytokine.

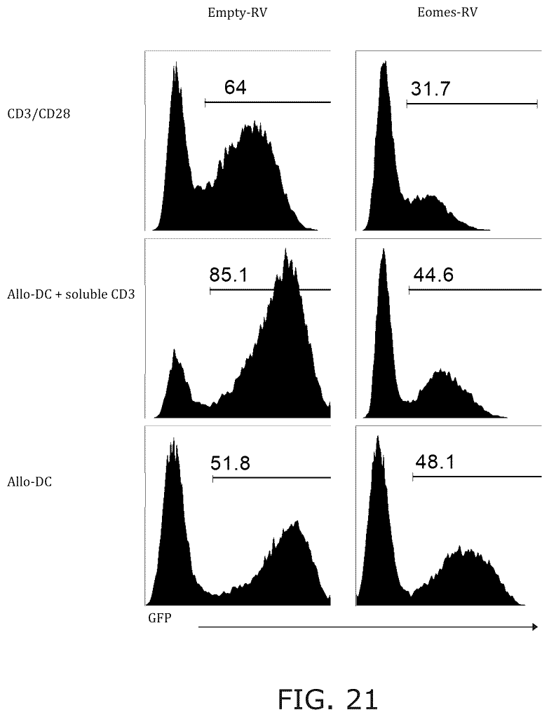

Next, the presented inventors generated alloantigen specific T_{R}1 cells by expression of a recombinant Eomes coding sequence in CD4⁺ T cells and stimulation of these cells with allogenic DC (allo-DC). Stimulation with allo-DC with or without concurrent use of soluble CD3 demonstrates similar efficiency of retroviral transduction compared to stimulation with plate bound CD3 and CD28 (FIG. 21). Retrovirally transduced CD4⁺ T cells will be FACS sorted to GFP^{hi} cells and polarized to T_{R}1 cells.

### Materials and Methods

Retrovirus production. Retrovirus production was performed as described before (59).

Retroviral transduction. Empty or Eomes expressing Retrovirus (RV) was centrifuged onto RetroNectin (Takara, 32 μg/mL)-coated non-tissue culture treated plates for 1 h at 3000 g at 4° C. B6.WT CD4⁺ T cells (purified with MACS selection) were stimulated with plate-bound CD3 (2C11, produced in house, 2.5-10 μg/mL) and CD28 (N3751, produced in house, 0.5 −1 ug/mL) for 20-24 hours, with allogenic dendritic cells (allo-DC, CD11c⁺MHCII⁺, MACS purified from spleens of B6D2F1 mice) and soluble CD3 (1 μg/mL) for 20-24 hours, or with allo-DC only for 48 hours before cultivating with the retrovirus in the presence of rhIL-2 (Aldesleukin, 100 U/mL) for 4-6 h. Cells were then washed and cultured in the presence of rhIL-2 (100 U/mL) for 3-4 days, FACS sorted to GFP^{hi} population and allowed for further expansion 1-2 days for in vitro generation of T_{R}1 populations.

Flow cytometry. Surface staining, intracellular cytokine staining, and flow cytometric analysis were performed as described before (59).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. M. G. Roncarolo et al., Interleukin-10-secreting type 1 regulatory T cells in rodents and humans. Immunological reviews 212, 28-50 (2006).
2. R. Bacchetta et al., High levels of interleukin 10 production in vivo are associated with tolerance in SCID patients transplanted with HLA mismatched hematopoietic stem cells. The Journal of experimental medicine 179, 493-502 (1994).
3. S. Gregori, K. S. Goudy, M. G. Roncarolo, The cellular and molecular mechanisms of immuno-suppression by human type 1 regulatory T cells. Frontiers in immunology 3, 30 (2012).
4. H. Groux et al., A CD4⁺ T-cell subset inhibits antigen-specific T-cell responses and prevents colitis. Nature 389, 737-742 (1997).
5. C. Pot, L. Apetoh, V. K. Kuchroo, Type 1 regulatory T cells (Tr1) in autoimmunity. Seminars in immunology 23, 202-208 (2011).
6. C. Neumann et al., Role of Blimp-1 in programing Th effector cells into IL-10 producers. The Journal of experimental medicine 211, 1807-1819 (2014).
7. M. Montes de Oca et al., Blimp-1-Dependent IL-10 Production by Tri Cells Regulates TNF-Mediated Tissue Pathology. PLoS pathogens 12, e1005398 (2016).
8. C. Heinemann et al., IL-27 and IL-12 oppose pro-inflammatory IL-23 in CD4⁺ T cells by inducing Blimp1. Nature communications 5, 3770 (2014).
9. N. Gagliani et al., Coexpression of CD49b and LAG-3 identifies human and mouse T regulatory type 1 cells. Nature medicine 19, 739-746 (2013).
10. L. Apetoh et al., The aryl hydrocarbon receptor interacts with c-Maf to promote the differentiation of type 1 regulatory T cells induced by IL-27. Nature immunology 11, 854-861 (2010).
11. A. Vasanthakumar, A. Kallies, IL-27 paves different roads to Tr1. European journal of immunology 43, 882-885 (2013).
12. A. Awasthi et al., A dominant function for interleukin 27 in generating interleukin 10-producing anti-inflammatory T cells. Nature immunology 8, 1380-1389 (2007).
13. A. Cope, G. Le Friec, J. Cardone, C. Kemper, The Th1 life cycle: molecular control of IFN-gamma to IL-10 switching. Trends in immunology 32, 278-286 (2011).
14. J. L. Ferrara, J. E. Levine, P. Reddy, E. Holler, Graft-versus-host disease. Lancet 373, 1550-1561 (2009).
15. B. R. Blazar, W. J. Murphy, M. Abedi, Advances in graft-versus-host disease biology and therapy. Nature reviews. Immunology 12, 443-458 (2012).
16. K. Matsuoka et al., Altered regulatory T cell homeostasis in patients with CD4⁺ lymphopenia following allogeneic hematopoietic stem cell transplantation. The Journal of clinical investigation 120, 1479-1493 (2010).
17. B. R. Blazar et al., Interleukin-10 dose-dependent regulation of CD4⁺ and CD8⁺ T cell-mediated graft-versus-host disease. Transplantation 66, 1220-1229 (1998).
18. E. S. Morris et al., Donor treatment with pegylated G-CSF augments the generation of IL-10-producing regulatory T cells and promotes transplantation tolerance. Blood 103, 3573-3581 (2004).
19. M. Kamanaka et al., Expression of interleukin-10 in intestinal lymphocytes detected by an interleukin-10 reporter knockin tiger mouse. Immunity 25, 941-952 (2006).
20. Y. Y. Wan, R. A. Flavell, Identifying Foxp3-expressing suppressor T cells with a bicistronic reporter. Proceedings of the National Academy of Sciences of the United States of America 102, 5126-5131 (2005).
21. E. Cretney et al., The transcription factors Blimp-1 and IRF4 jointly control the differentiation and function of effector regulatory T cells. Nature immunology 12, 304-311 (2011).
22. L. Gabrysova et al., Negative feedback control of the autoimmune response through antigen-induced differentiation of IL-10-secreting Th1 cells. The Journal of experimental medicine 206, 1755-1767 (2009).
23. F. J. Quintana et al., Control of T(reg) and T(H)17 cell differentiation by the aryl hydrocarbon receptor. Nature 453, 65-71 (2008).
24. I. D. Mascanfroni et al., Metabolic control of type 1 regulatory T cell differentiation by AHR and HIF1-alpha. Nature medicine 21, 638-646 (2015).
25. E. Zigmond et al., Macrophage-restricted interleukin-10 receptor deficiency, but not IL-10 deficiency, causes severe spontaneous colitis. Immunity 40, 720-733 (2014).
26. G. R. Hill et al., Total body irradiation and acute graft-versus-host disease: the role of gastrointestinal damage and inflammatory cytokines. Blood 90, 3204-3213 (1997).
27. K. A. Markey, K. P. MacDonald, G. R. Hill, The biology of graft-versus-host disease: experimental systems instructing clinical practice. Blood 124, 354-362 (2014).
28. J. S. Stumhofer et al., A role for IL-27p28 as an antagonist of gp130-mediated signaling. Nature immunology 11, 1119-1126 (2010).
29. J. M. Kim, J. P. Rasmussen, A. Y. Rudensky, Regulatory T cells prevent catastrophic autoimmunity throughout the lifespan of mice. Nature immunology 8, 191-197 (2007).
30. A. McNally, G. R. Hill, T. Sparwasser, R. Thomas, R. J. Steptoe, CD4⁺CD25⁺ regulatory T cells control CD8⁺ T-cell effector differentiation by modulating IL-2 homeostasis. Proceedings of the National Academy of Sciences of the United States of America 108, 7529-7534 (2011).
31. K. Lahl et al., Selective depletion of Foxp3⁺ regulatory T cells induces a scurfy-like disease. The Journal of experimental medicine 204, 57-63 (2007).
32. H. M. Shin et al., Epigenetic modifications induced by Blimp-1 Regulate CD8(+) T cell memory progression during acute virus infection. Immunity 39, 661-675 (2013).
33. A. O'Garra, P. Vieira, T(H)1 cells control themselves by producing interleukin-10. Nature reviews. Immunology 7, 425-428 (2007).
34. S. M. Gordon et al., The transcription factors T-bet and Eomes control key checkpoints of natural killer cell maturation. Immunity 36, 55-67 (2012).
35. A. M. Intlekofer et al., Effector and memory CD8⁺ T cell fate coupled by T-bet and eomesodermin. Nature immunology 6, 1236-1244 (2005).
36. B. J. Raveney et al., Eomesodermin-expressing T-helper cells are essential for chronic neuroinflammation. Nature communications 6, 8437 (2015).
37. M. A. Curran et al., Systemic 4-1BB activation induces a novel T cell phenotype driven by high expression of Eomesodermin. The Journal of experimental medicine 210, 743-755 (2013).
38. E. Lupar et al., Eomesodermin Expression in CD4⁺ T Cells Restricts Peripheral Foxp3 Induction. Journal of immunology 195, 4742-4752 (2015).
39. K. Ichiyama et al., Transcription factor Smad-independent T helper 17 cell induction by transforming-growth factor-beta is mediated by suppression of eomesodermin. Immunity 34, 741-754 (2011).
40. N. Gagliani et al., Th17 cells transdifferentiate into regulatory T cells during resolution of inflammation. Nature 523, 221-225 (2015).
41. P. P. Ahern et al., Interleukin-23 drives intestinal inflammation through direct activity on T cells. Immunity 33, 279-288 (2010).
42. M. J. McGeachy et al., TGF-beta and IL-6 drive the production of IL-17 and IL-10 by T cells and restrain T(H)-17 cell-mediated pathology. Nature immunology 8, 1390-1397 (2007).
43. D. Jankovic, D. G. Kugler, A. Sher, IL-10 production by CD4⁺ effector T cells: a mechanism for self-regulation. Mucosal immunology 3, 239-246 (2010).
44. M. Batten et al., Interleukin 27 limits autoimmune encephalomyelitis by suppressing the development of interleukin 17-producing T cells. Nature immunology 7, 929-936 (2006).
45. L. Belle et al., Blockade of interleukin 27 signaling reduces GVHD in mice by augmenting Treg reconstitution and stabilizing FOXP3 expression. Blood, (2016).
46. G. A. Kennedy et al., Addition of interleukin-6 inhibition with tocilizumab to standard graft-versus-host disease prophylaxis after allogeneic stem-cell transplantation: a phase ½ trial. The Lancet. Oncology 15, 1451-1459 (2014).
47. V. Lazarevic, L. H. Glimcher, G. M. Lord, T-bet: a bridge between innate and adaptive immunity. Nature reviews. Immunology 13, 777-789 (2013).
48. F. Cruz-Guilloty et al., Runx3 and T-box proteins cooperate to establish the transcriptional program of effector CTLs. The Journal of experimental medicine 206, 51-59 (2009).
49. Y. Iwasaki et al., Egr-2 transcription factor is required for Blimp-1-mediated IL-10 production in IL-27-stimulated CD4⁺ T cells. European journal of immunology 43, 1063-1073 (2013).
50. A. Xin et al., A molecular threshold for effector CD8(+) T cell differentiation controlled by transcription factors Blimp-1 and T-bet. Nature immunology 17, 422-432 (2016).
51. A. Kallies, A. Xin, G. T. Belz, S. L. Nutt, Blimp-1 transcription factor is required for the differentiation of effector CD8(+) T cells and memory responses. Immunity 31, 283-295 (2009).
52. A. Kallies et al., Plasma cell ontogeny defined by quantitative changes in blimp-1 expression. The Journal of experimental medicine 200, 967-977 (2004).
53. M. C. Pils et al., Monocytes/macrophages and/or neutrophils are the target of IL-10 in the LPS endotoxemia model. European journal of immunology 40, 443-448 (2010).
54. A. Roers et al., T cell-specific inactivation of the interleukin 10 gene in mice results in enhanced T cell responses but normal innate responses to lipopolysaccharide or skin irritation. The Journal of experimental medicine 200, 1289-1297 (2004).
55. E. E. Kara et al., CCR2 defines in vivo development and homing of IL-23-driven GM-CSF-producing Th17 cells. Nature communications 6, 8644 (2015).
56. P. Zhang et al., Induced regulatory T cells promote tolerance when stabilized by rapamycin and IL-2 in vivo. Journal of immunology 191, 5291-5303 (2013).
57. A. C. Burman et al., IFNgamma differentially controls the development of idiopathic pneumonia syndrome and GVHD of the gastrointestinal tract. Blood 110, 1064-1072 (2007).
58. M. Koyama et al., Donor colonic CD103⁺ dendritic cells determine the severity of acute graft-versus-host disease. J Exp Med 212, 1303-1321 (2015).
59. P. Zhang et al. Eomesodermin promotes the development of type 1 regulatory T (T_{R}1) cells. Sci Immunol 2017; 2(10). doi: 10.1126/sciimmunol.aah7152
60. A. Vasanthakumar et al. IL-27 paves different roads to Tr1. European journal of immunology 2013; 43(4): 882-885. doi: 10.1002/eji.201343479

What is claimed is:

1. A population of immunosuppressive regulatory T cells comprising Eomes⁺IL-10⁺ CD4⁺ T cells that comprise a construct comprising an Eomes coding sequence in operable connection with a regulatory sequence that is operable in the T cells.

2. The population of claim 1, wherein the Eomes⁺IL-10⁺ CD4⁺ T cells comprise Eomes^{hi} T cells.

3. The population of claim 1, wherein the Eomes⁺IL-10⁺ CD4⁺ T cells comprise T-bet^{lo} T cells.

4. The population of claim 1, wherein the Eomes⁺IL-10⁺ CD4⁺ T cells comprise IFNγ⁺ T cells.

5. The population of claim 1, wherein the Eomes⁺IL-10⁺ CD4⁺ T cells are high for IL-10.

6. The population of claim 1, wherein the Eomes⁺IL-10⁺ CD4⁺ T cells are high for IL-IFNγ.

7. The population of claim 1, wherein the Eomes⁺IL-10⁺ CD4⁺ T cells are high for IL-10 and IL-IFNγ.

8. The population of claim 1, wherein the Eomes⁺IL-10⁺ CD4⁺ T cells are negative or low for T_{H}2 cytokines.

9. The population of claim 8, wherein the T_{H}2 cytokines are selected from the group consisting of IL-4, IL-13 and IL-5.

10. The population of claim 1, wherein the Eomes⁺IL-10⁺ CD4⁺ T cells are negative or low for one or more T_{H}17 cytokines.

11. The population of claim 10, wherein the T_{H}17 cytokines are selected from the group consisting of IL-17, IL-6 and GM-CSF.

12. The population of claim 1, wherein the Eomes⁺IL-10⁺ CD4⁺ T cells are capable of suppressing IL-2 production.

13. The population of claim 1, wherein the Eomes⁺IL-10⁺ CD4⁺ T cells are capable of suppressing cell proliferation.

14. The population of claim 1, wherein the Eomes⁺IL-10⁺ CD4⁺ T cells are capable of suppressing cell migration.

15. The population of claim 1, wherein the Eomes⁺IL-10⁺ CD4⁺ T cells are capable of suppressing cytokine production.

16. The population of claim 1, wherein the Eomes⁺IL-10⁺ CD4⁺ T cells are capable of suppressing T cell effector function.

17. The population of claim 1, wherein the Eomes⁺IL-10⁺ CD4⁺ T cells are capable of suppressing T cell killing.

18. The population of claim 1, wherein the Eomes⁺IL-10⁺ CD4⁺ T cells are capable of suppressing T cell proliferation.
